(19) United States
(12) Patent Application Publication
(10) Pub. No.: US 2022/0195393 A1
(43) Pub. Date: Jun. 23, 2022

(54) METHODS FOR STORAGE OF STEM CELLS

(71) Applicant: Ossium Health, Inc., San Francisco, CA (US)

(72) Inventors: Erik J. WOODS, Carmel, IN (US); Hannah Marie MILLER, Speedway, IN (US); Brian H. JOHNSTONE, Fishers, IN (US)

(21) Appl. No.: 17/667,434

(22) Filed: Feb. 8, 2022

Related U.S. Application Data
(63) Continuation of application No. PCT/US2021/064433, filed on Dec. 20, 2021.
(60) Provisional application No. 63/128,780, filed on Dec. 21, 2020.

(51) Int. Cl. C12N 5/0775

(52) U.S. Cl. CPC C12N 5/0663; C12N 2523/00

(57) ABSTRACT

Described herein are methods and systems for preparing samples of stem cells for direct infusion. Some of the methods and systems described herein comprise hypothermic storage of the stem cells. The methods and systems described herein can supplement shipment of stem cells across long distances such that the stem cells are prepared for direct infusion upon receipt of the cells.

### CROSS-REFERENCE

This application is a continuation of International Application No. PCT/US2021/064433, filed Dec. 20, 2021, which claims the benefit of U.S. Provisional Application No. 63/128,780, filed Dec. 21, 2020, which are incorporated herein by reference in their entirety.

### BACKGROUND

Induction of immune tolerance with solid organ and vascular composite allografts is the gold standard for transplantation medicine. Induction of immune tolerance to mismatched grafts would obviate the need for life-long immunosuppression which is associated with serious adverse outcomes, such as renal failure, cancers, and infections. Currently the most promising means of tolerance induction is through establishing a mixed chimeric state by transplantation of donor hematopoietic stem cells; however, with the exception of tolerogenic organs such as kidneys, the mixed chimerism approach has not achieved durable immune tolerance in preclinical or clinical trials with most solid organs or vascular composite allotransplants (VCA).

Mesenchymal stem (stromal) cells (MSCs) have been identified as potentially useful adjuvant to stem cell transplants (SCT) for promoting mixed chimerism as well as promoting complementary peripheral immunomodulatory functions, along with solid organ transplants (SOT). However, there are many unresolved issues to address before clinical translation of these promising therapeutic cells. A primary impediment is the source of MSCs, which are rare in all tissues and require invasive procedures for procurement. Low abundance mandates extensive expansion in culture to generate sufficient numbers for human dosing. It has been observed in the clinical setting that the degree of expansion is negatively correlated with outcomes. It has been further observed that cryopreservation of MSCs is also negatively correlated with outcomes. Therefore, there exists a need for methods of hypothermic storage of MSC populations which retain their viability to enable their distribution for clinically useful applications.

### SUMMARY

Provided herein, in one aspect, is a method for preparing stem cells for infusion, the method comprising: (a) providing a cryopreserved population of cells comprising said stem cells; (b) warming said stem cells to a first temperature and holding said stem cells at said first temperature for a first period of time; and (c) changing said first temperature to a second temperature and maintaining said stem cells at said second temperature for a period of time.

An aspect of the present disclosure is a method for preparing stem cells for infusion, the method comprising: providing a cryopreserved population of cells comprising the stem cells; warming the stem cells to a first temperature and holding the stem cells at the first temperature for a first period of time; and changing the first temperature to a second temperature and maintaining the stem cells at the second temperature for a period of time. In some embodiments, the first temperature is greater than 0° C. In some embodiments, the second temperature is less than 40° C. In some embodiments, the second temperature is hypothermic. In some embodiments, the first period of time or the time period is less than one week. In some embodiments, the first period of time or the time period is less than 5 days. In some embodiments, the first period of time or the time period is less than 2 days. In some embodiments, the first period of time or the time period is less than 1 day. In some embodiments, the first period of time or the time period is less than 12 hours. In some embodiments, the first period of time or the time period is less than 6 hours. In some embodiments, the first period of time or the time period is less than 2 hours. In some embodiments, the method further comprises, prior to providing a cryopreserved population of cells comprising the stem cells, the stem cells were passaged. In some embodiments, the method further comprises, prior to providing a cryopreserved population of cells comprising the stem cells, the stem cells were passaged at least one time. In some embodiments, the method further comprises, prior to providing a cryopreserved population of cells comprising the stem cells, the stem cells were passaged at least 2 times. In some embodiments, the method further comprises, prior to providing a cryopreserved population of cells comprising the stem cells, the stem cells were passaged at least 4 times. In some embodiments, the method further comprises, prior to changing the first temperature to a second temperature and maintaining the stem cells at the second temperature for a period of time, culturing the stem cells at about 10,000 cells/cm2 to about 50,000 cells/cm2 at the first temperature. In some embodiments, the method further comprises, prior to changing the first temperature to a second temperature and maintaining the stem cells at the second temperature for a period of time, culturing the stem cells at about 32,000 cells/cm2 at the first temperature. In some embodiments, the method further comprises, prior to changing the first temperature to a second temperature and maintaining the stem cells at the second temperature for a period of time, packaging the stem cells in a volume comprising about 10×102 live cells/mL to about 10×1010 live cell s/mL and maintaining the stem cells at the second temperature. In some embodiments, the method further comprises, prior to changing the first temperature to a second temperature and maintaining the stem cells at the second temperature for a period of time, packaging the stem cells in a volume comprising about 10×106 live cells/mL and maintaining the stem cells at the second temperature. In some embodiments, the method further comprises, prior to changing the first temperature to a second temperature and maintaining the stem cells at the second temperature for a period of time, packaging the stem cells in a volume comprising less than 10 mL, 9 mL, 8 mL, 7 mL, or 6 mL. In some embodiments, the method further comprises, prior to changing the first temperature to a second temperature and maintaining the stem cells at the second temperature for a period of time, packaging the stem cells in a volume comprising 5 mL. In some embodiments, the stem cells are held at the first temperature in a medium comprising one or more amino acids. In some embodiments, the medium comprising one or more amino acids further comprises one or more vitamins. In some embodiments, the medium comprising one or more amino acids further comprises Table 1. In some embodiments, the first time period is a time period wherein the stem cells do not double. In some embodiments, the first time period is a time period wherein the stem cells undergo one or more population doublings. In some embodiments, the stem cells are maintained to less than 80% confluency relative to a cell culture storage medium during the first time period or the time period. In some embodiments, the stem cells are maintained at the second temperature in a rinse media, wherein the rinse media comprises an electrolyte formulation, human serum albumin (HSA), or both. In some embodiments, the stem cells comprise vertebral bone adherent mesenchymal stem cells (vBA-MSCs), vertebral body bone marrow mesenchymal stem cells (vBM-MSCs), or both. In some embodiments, the stem cells are derived from a cadaver. In some embodiments, the stem cells comprise at least about 10,000 to about 300,000 CFU-F/1 million viable cells. In some embodiments, the stem cells comprise less than about 1% to about 10% CD45+ cells.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at less than 40° C. In some embodiments, the cell culture was passaged at least one time. In some embodiments, the method further comprises, subsequent to thawing the cell culture, the cell culture is not doubled. In some embodiments, the method further comprises, subsequent to thawing the cell culture, the cell culture is maintained to undergo one or more population doublings. In some embodiments, the method further comprises, subsequent to thawing the cell culture, the cell culture is maintained to less than 80% confluency relative to a cell culture storage medium. In some embodiments, the cell culture is maintained in a medium comprising one or more amino acids. In some embodiments, the medium comprising one or more amino acids further comprises one or more vitamins. In some embodiments, the medium comprising one or more amino acids further comprises Table 1. In some embodiments, the cell culture was passaged at least 2 times, at least 3 times, at least 4 times, at least 5 times, at least 6 times, at least 7 times, at least 8 times, at least 9 times, or at least 10 times. In some embodiments, the cell culture was cryopreserved in a cryopreservation media, wherein the cryopreservation media comprises an electrolyte formulation, human serum albumin (HSA), dimethyl sulfoxide (DMSO), or any combination thereof. In some embodiments, the cryopreservation media comprises about 1% to about 5% HSA. In some embodiments, the cryopreservation media comprises about 2.5% HSA. In some embodiments, the cryopreservation media comprises about 1% to about 10% DMSO. In some embodiments, the cryopreservation media comprises about 5% DMSO. In some embodiments, the electrolyte formulation is Plasmalyte A. In some embodiments, the thawing occurs at about 30° C. to about 40° C. In some embodiments, the thawing occurs at about 37° C. In some embodiments, the method further comprises resuspending the cell culture in a rinse media, wherein the rinse media comprises an electrolyte formulation, human serum albumin (HSA), or both; and maintaining the cell culture in less than room temperature. In some embodiments, the method further comprises, prior to (c), resuspending the cell culture in a rinse media, wherein the rinse media comprises an electrolyte formulation, human serum albumin (HSA), or both. In some embodiments, the rinse media is fresh. In some embodiments, the rinse media comprises about 1% to about 5% HSA. In some embodiments, the rinse media comprises about 2.5% HSA. In some embodiments, the electrolyte formulation is Plasmalyte A. In some embodiments, the cell culture is maintained at less than room temperature for at least about 30 minutes. In some embodiments, the cell culture is maintained at less than room temperature for at least 12 hours. In some embodiments, the cell culture is maintained at less than room temperature for at least 24 hours. In some embodiments, the cell culture is maintained at less than room temperature for at least 48 hours. In some embodiments, the cell culture is maintained at less than 37° C. In some embodiments, the cell culture is maintained at less than 35° C. In some embodiments, the cell culture is maintained at less than 30° C. In some embodiments, the method further comprises, subsequent to (b) the cell culture is maintained at hypothermic conditions. In some embodiments, the cell culture is maintained at less than 25° C. In some embodiments, the cell culture is maintained at less than 20° C. In some embodiments, the cell culture is maintained at about 2° C. to about 8° C. In some embodiments, the method further comprises, culturing the cell culture at about 10,000 cells/cm2 to about 50,000 cells/cm2. In some embodiments, the method further comprises, culturing the cell culture at about 32,000 cells/cm2. In some embodiments, the method further comprises, packaging the cell culture in a volume comprising about 10×102 live cells/mL to about 10×1010 live cells/mL. In some embodiments, the method further comprises, packaging the cell culture in a volume comprising about 10×106 live cells/mL. In some embodiments, the method further comprises, packaging the cell culture in a volume comprising less than 10 mL, 9 mL, 8 mL, 7 mL, or 6 mL. In some embodiments, the method further comprises, packaging the cell culture in a volume comprising 5 mL. In some embodiments, the sample of stem cells comprises at least about 70% viable cells. In some embodiments, the cell culture comprises vertebral bone adherent mesenchymal stem cells (vBA-MSCs), vertebral body bone marrow mesenchymal stem cells (vBM-MSCs), or both. In some embodiments, the cell culture is derived from a cadaver. In some embodiments, the cell culture comprises at least about 10,000 to about 300,000 CFU-F/1 million viable cells. In some embodiments, the cell culture comprises less than about 1% to about 10% CD45+ cells.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at greater than 0° C. In some embodiments, the sample of stem cells comprises at least about 70% viable cells after maintaining the cell culture at greater than 0° C.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at less than room temperature; wherein the sample of stem cells comprises at least about 70% viable cells. In some embodiments, the sample of stem cells comprises at least about 70% viable cells after maintaining the cell culture at greater than 0° C.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at less than room temperature; wherein the sample of stem cells comprises at least about 70% viable cells. In some embodiments, the sample of stem cells comprises at least about 70% viable cells after maintaining the cell culture at greater than 0° C.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at less than room temperature; wherein the sample of stem cells comprises less than about 1% to about 10% CD45+ cells. In some embodiments, the sample of stem cells comprises less than about 5% CD45+ cells. In some embodiments, the sample of stem cells comprises less than about 1% to about 10% CD35+ cells. In some embodiments, the sample of stem cells comprises less than about 5% CD35+ cells.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at less than room temperature; wherein the sample of stem cells comprises at least about 90% to about 99% CD90+ cells. In some embodiments, the sample of stem cells comprises at least about 95% CD90+ cells.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at less than room temperature; wherein the sample of stem cells comprises at least about 90% to about 99% CD105+ cells. In some embodiments, the sample of stem cells comprises at least about 95% CD105+ cells. In some embodiments, the sample of stem cells comprises at least about 70% viable cells after maintaining the cell culture at greater than 0° C.

Another aspect of the present disclosure is a method for preparing a sample of stem cells for direct infusion, the method comprising: providing a cell culture comprising the sample of stem cells wherein the cell culture was cryopreserved and passaged; thawing the cell culture; and maintaining the cell culture at less than room temperature; wherein the sample of stem cells comprises at least about 10,000 to about 300,000 CFU-F/1 million viable cells. In some embodiments, the sample of stem cells comprises at least about 10,000 CFU-F/1 million viable cells. In some embodiments, the sample of stem cells comprises at least about 70% viable cells after maintaining the cell culture at greater than 0° C. In some embodiments, the sample of stem cells comprise vertebral bone adherent mesenchymal stem cells (vBA-MSCs), vertebral body bone marrow mesenchymal stem cells (vBM-MSCs), or both. In some embodiments, the method further comprises, subsequent to thawing the cell culture, the cell culture is not doubled. In some embodiments, the method further comprises, subsequent to thawing the cell culture, the cell culture is maintained to undergo one or more population doublings. In some embodiments, the method further comprises, subsequent to thawing the cell culture, the cell culture is maintained to less than 80% confluency relative to a cell culture storage medium. In some embodiments, the cell culture is maintained in a medium comprising one or more amino acids. In some embodiments, the medium comprising one or more amino acids further comprises one or more vitamins. In some embodiments, the medium comprising one or more amino acids further comprises Table 1. In some embodiments, the cell culture was passaged at least 2 times, at least 3 times, at least 4 times, at least 5 times, at least 6 times, at least 7 times, at least 8 times, at least 9 times, or at least 10 times. In some embodiments, the cell culture was cryopreserved in a cryopreservation media, wherein the cryopreservation media comprises an electrolyte formulation, human serum albumin (HSA), dimethyl sulfoxide (DMSO), or any combination thereof. In some embodiments, the cryopreservation media comprises about 1% to about 5% HSA. In some embodiments, the cryopreservation media comprises about 2.5% HSA. In some embodiments, the cryopreservation media comprises about 1% to about 10% DMSO. In some embodiments, the cryopreservation media comprises about 5% DMSO. In some embodiments, the electrolyte formulation is Plasmalyte A. In some embodiments, the thawing occurs at about 30° C. to about 40° C. In some embodiments, the thawing occurs at about 37° C. In some embodiments, the method further comprises resuspending the cell culture in a rinse media, wherein the rinse media comprises an electrolyte formulation, human serum albumin (HSA), or both; and maintaining the cell culture in less than room temperature. In some embodiments, the method further comprises, prior to (c), resuspending the cell culture in a rinse media, wherein the rinse media comprises an electrolyte formulation, human serum albumin (HSA), or both. In some embodiments, the rinse media is fresh. In some embodiments, the rinse media comprises about 1% to about 5% HSA. In some embodiments, the rinse media comprises about 2.5% HSA. In some embodiments, the electrolyte formulation is Plasmalyte A. In some embodiments, the cell culture is maintained at less than room temperature for at least about 30 minutes. In some embodiments, the cell culture is maintained at less than room temperature for at least 12 hours. In some embodiments, the cell culture is maintained at less than room temperature for at least 24 hours. In some embodiments, the cell culture is maintained at less than room temperature for at least 48 hours. In some embodiments, the cell culture is maintained at less than 37° C. In some embodiments, the cell culture is maintained at less than 35° C. In some embodiments, the cell culture is maintained at less than 30° C. In some embodiments, the method further comprises, subsequent to thawing the cell culture, the cell culture is maintained at hypothermic conditions. In some embodiments, the cell culture is maintained at less than 25° C. In some embodiments, the cell culture is maintained at less than 20° C. In some embodiments, the cell culture is maintained at about 2° C. to about 8° C. In some embodiments, the method further comprises, culturing the cell culture at about 10,000 cells/cm2 to about 50,000 cells/cm2. In some embodiments, the method further comprises, culturing the cell culture at about 32,000 cells/cm2. In some embodiments, the method further comprises, packaging the cell culture in a volume comprising about 10×102 live cells/mL to about 10×1010 live cells/mL. In some embodiments, the method further comprises, packaging the cell culture in a volume comprising about 10×106 live cells/mL. In some embodiments, the method further comprises, packaging the cell culture in a volume comprising less than 10 mL, 9 mL, 8 mL, 7 mL, or 6 mL. In some embodiments, the method further comprises, packaging the cell culture in a volume comprising 5 mL. In some embodiments, the sample of stem cells comprises at least about 70% viable cells. In some embodiments, the cell culture comprises vertebral bone adherent mesenchymal stem cells (vBA-MSCs), vertebral body bone marrow mesenchymal stem cells (vBM-MSCs), or both. In some embodiments, the cell culture is derived from a cadaver.

In some embodiments, said first temperature is greater than 0° C. In some embodiments, said second temperature is less than 40° C. In some embodiments, said second temperature is hypothermic.

In some embodiments, said time period is less than one week. In some embodiments, said time period is less than 5 days. In some embodiments, said time period is less than 2 days. In some embodiments, said time period is less than 1 day. In some embodiments, said time period is less than 12 hours. In some embodiments, said time period is less than 6 hours. In some embodiments, said time period is less than 2 hours.

In some embodiments, prior to (a), said stem cells were passaged. In some embodiments, prior to (a), said stem cells were passaged at least one time. In some embodiments, prior to (a), said stem cells were passaged at least 2 times. In some embodiments, prior to (a), said stem cells were passaged at least 4 times.

In some embodiments, the method further comprises, prior to (c), culturing said stem cells at about 10,000 cells/cm² to about 50,000 cells/cm² at said first temperature. In some embodiments, the method further comprises, prior to (c), culturing said stem cells at about 32,000 cells/cm² at said first temperature.

In some embodiments, the method further comprises, prior to (c), packaging said stem cells in a volume comprising about 10×10² live cells/mL to about 10×10¹⁰ live cells/mL and maintaining said stem cells at said second temperature. In some embodiments, the method further comprises, prior to (c), packaging said stem cells in a volume comprising about 10×10⁶ live cells/mL and maintaining said stem cells at said second temperature.

In some embodiments, the method further comprises, prior to (c), packaging said stem cells in a volume comprising less than 10 mL, 9 mL, 8 mL, 7 mL, or 6 mL. In some embodiments, the method further comprises, prior to (c), packaging said stem cells in a volume comprising 5 mL.

Provided herein, in another aspect, is a method for preparing a sample of stem cells for direct infusion, the method comprising: (a) providing a cell culture comprising said sample of stem cells wherein said cell culture was cryopreserved and passaged; (b) thawing said cell culture; and (c) maintaining said cell culture at less than 40° C.

In some embodiments, said cell culture was passaged at least one time. In some embodiments, subsequent to (b) said cell culture is not doubled. In some embodiments, said cell culture was passaged at least 2 times, at least 3 times, at least 4 times, at least 5 times, at least 6 times, at least 7 times, at least 8 times, at least 9 times, or at least 10 times.

In some embodiments, said cell culture is maintained at less than 37° C. The method of any one of the preceding claims, wherein said cell culture is maintained at less than 35° C. In some embodiments, said cell culture is maintained at less than 30° C. In some embodiments, subsequent to (b) said cell culture is maintained at hypothermic conditions. In some embodiments, said cell culture is maintained at less than 25° C. In some embodiments, said cell culture is maintained at less than 20° C. In some embodiments, said cell culture is maintained at about 2° C. to about 8° C.

In some embodiments, said cell culture was cryopreserved in a cryopreservation media, wherein said cryopreservation media comprises an electrolyte formulation, human serum albumin (HSA), dimethyl sulfoxide (DMSO), or any combination thereof. In some embodiments, said cryopreservation media comprises about 1% to about 5% HSA. In some embodiments, said cryopreservation media comprises about 2.5% HSA. In some embodiments, said cryopreservation media comprises about 1% to about 10% DMSO. In some embodiments, said cryopreservation media comprises about 5% DMSO. In some embodiments, said electrolyte formulation is Plasmalyte A.

In some embodiments, said thawing occurs at about 30° C. to about 40° C. In some embodiments, said thawing occurs at about 37° C.

In some embodiments, said cell culture is maintained at less than room temperature for at least about 30 minutes. In some embodiments, said cell culture is maintained at less than room temperature for at least 12 hours. In some embodiments, said cell culture is maintained at less than room temperature for at least 24 hours. In some embodiments, said cell culture is maintained at less than room temperature for at least 48 hours.

In some embodiments, the method further comprises, prior to (c), resuspending said cell culture in a rinse media, wherein said rinse media comprises an electrolyte formulation, human serum albumin (HSA), or both. In some embodiments, the method further comprises, prior to (c), resuspending said cell culture in a rinse media, wherein said rinse media comprises an electrolyte formulation, human serum albumin (HSA), or both. In some embodiments, said rinse media is fresh. In some embodiments, said rinse media comprises about 1% to about 5% HSA. In some embodiments, said rinse media comprises about 2.5% HSA. In some embodiments, said electrolyte formulation is Plasmalyte A.

In some embodiments, the method further comprises, prior to (c), culturing said cell culture at about 10,000 cells/cm² to about 50,000 cells/cm². In some embodiments, the method further comprises, prior to (c), culturing said cell culture at about 32,000 cells/cm².

In some embodiments, the method further comprises, prior to (c), packaging said cell culture in a volume comprising about 10×10² live cells/mL to about 10×10¹⁰ live cells/mL. In some embodiments, the method further comprises, prior to (c), packaging said cell culture in a volume comprising about 10×10⁶ live cells/mL.

In some embodiments, the method further comprises, prior to (c), packaging said cell culture in a volume comprising less than 10 mL, 9 mL, 8 mL, 7 mL, or 6 mL. In some embodiments, the method further comprises, prior to (c), packaging said cell culture in a volume comprising 5 mL.

In some embodiments, said sample of stem cells comprises at least about 70% viable cells after (c).

Provided herein, in another aspect, is a method for preparing a sample of stem cells for direct infusion, the method comprising: (a) providing a cell culture comprising said sample of stem cells wherein said cell culture was cryopreserved and passaged; (b) thawing said cell culture; and (c) maintaining said cell culture at greater than 0° C.

In some embodiments, said sample of stem cells comprises at least about 70% viable cells after (c).

Provided herein, in another aspect, is a method for preparing a sample of stem cells for direct infusion, the method comprising: (a) providing a cell culture comprising said sample of stem cells wherein said cell culture was cryopreserved and passaged; (b) thawing said cell culture; and (c) maintaining said cell culture at less than room temperature;

wherein said sample of stem cells comprises at least about 70% viable cells.

In some embodiments, said sample of stem cells comprises at least about 70% viable cells after (c).

Provided herein, in another aspect, is a method for preparing a sample of stem cells for direct infusion, the method comprising: (a) providing a cell culture comprising said sample of stem cells wherein said cell culture was cryopreserved and passaged; (b) thawing said cell culture; and (c) maintaining said cell culture at less than room temperature;

wherein said sample of stem cells comprises less than about 1% to about 10% CD45+ cells.

In some embodiments, said sample of stem cells comprises less than about 5% CD45+ cells.

In some embodiments, said sample of stem cells comprises less than about 1% to about 10% CD34+ cells. In some embodiments, said sample of stem cells comprises less than about 5% CD34+ cells.

Provided herein, in another aspect, is a method for preparing a sample of stem cells for direct infusion, the method comprising: (a) providing a cell culture comprising said sample of stem cells wherein said cell culture was cryopreserved and passaged; (b) thawing said cell culture; and (c) maintaining said cell culture at less than room temperature;

wherein said sample of stem cells comprises at least about 90% to about 99% CD90+ cells.

In some embodiments, said sample of stem cells comprises at least about 95% CD90+ cells.

Provided herein, in another aspect, is a method for preparing a sample of stem cells for direct infusion, the method comprising: (a) providing a cell culture comprising said sample of stem cells wherein said cell culture was cryopreserved and passaged; (b) thawing said cell culture; and (c) maintaining said cell culture at less than room temperature;

wherein said sample of stem cells comprises at least about 90% to about 99% CD105+ cells.

In some embodiments, said sample of stem cells comprises at least about 95% CD105+ cells.

In some embodiments, said sample of stem cells comprises at least about 70% viable cells after (c).

Provided herein, in another aspect, is a method for preparing a sample of stem cells for direct infusion, the method comprising: (a) providing a cell culture comprising said sample of stem cells wherein said cell culture was cryopreserved and passaged; (b) thawing said cell culture; and (c) maintaining said cell culture at less than room temperature; wherein said sample of stem cells comprises at least about 10,000 to about 300,000 CFU-F/1 million viable cells.

In some embodiments, said sample of stem cells comprises at least about 10,000 CFU-F/1 million viable cells.

In some embodiments, said sample of stem cells comprises at least about 70% viable cells after (c).

In some embodiments, said sample of stem cells comprise vertebral bone adherent mesenchymal stem cells (vBA-MSCs), vertebral body bone marrow mesenchymal stem cells (vBM-MSCs), or both.

Provided herein, in another aspect, is a method for preparing a sample of vertebral bone adherent mesenchymal stem cells (vBA-MSCs) for direct infusion, the method comprising: (a) providing a cell culture comprising said sample of stem cells wherein said cell culture was cryopreserved and passaged; (b) thawing said cell culture; and (c) maintaining said cell culture at less than room temperature.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the number of fibroblast colony-forming units (CFU-F) per 1 million cells plated 24 hours and 48 hours post-plating.

### DETAILED DESCRIPTION OF THE INVENTION

Typically, patients undergoing lifesaving procedures of stem cell transplants (SCT), solid organ transplants (SOT), and vascular composition allotransplants (VCA) are subjected to a life-long requirement of immune suppressing drugs that prevent their body from rejecting the organ or tissue graft. Unfortunately, immunosuppression drugs have been found to be associated with severe medical issues such as cancer, kidney failure and infections. Therefore, new medical procedures that reduce or overcome the need for immunosuppressive drugs are greatly needed. Previously deceased donor vertebral bodies (VB) bone marrow (BM) SCT from matched diseased organ donors have been found to induce immune tolerance following SOT and VCA. It is suggested that immunosuppression induction from transplanted VB BM STC rely on establishing mixed chimerism by SCT. It has also been suggested that MSCs promote stem cell engraftment in the BM and induce FoxP3+ TREG cell expansion as wells as possess additional synergistic immunomodulatory properties. Additionally, MSCs have been shown to possess potent immune system modulating activities which has contributed to widespread testing in clinical trials for various diseases associated with immune system dysfunction. Unfortunately, MSCs are extremely hard to obtain and subsequently have to be grown in a laboratory to generate sufficient numbers required to treat typical adult patients. To address this shortcoming, the systems and methods disclosed herein provide a needed complement to existing MSC and BM sources and extraction methodologies capable of producing MSCs and BM in sufficient quantity for the aforementioned applications.

The resulting processing steps mean that MSCs need to be further prepared before direct infusion into a subject. In practice, sites where the direct infusion of MSCs take place do not have personnel trained in MSC sample prep. Therefore there is a need for methods and systems of MSC sample preparation for direct infusion, wherein the MSCs do not require further processing steps upon arrival to the site of the direct infusion.

The methods and systems disclosed herein enable the hypothermic storage of large quantities of MSCs from human vertebral bone termed (vBA-MSCs) and Vertebral Body Bone Marrow Mesenchymal Stem Cells (vBM-MSCs) to allow for their distribution for clinical applications.

The methods and systems disclosed herein further enable the sample preparation of MSCs for direct infusion. In some embodiments, the methods and systems for sample preparation described herein do not require further processing steps at the site of infusion. In some embodiments, the methods and systems described herein provide a sample of MSCs wherein the MSCs are present in the sample comprising qualities suitable for direct infusion into a subject. Some of these qualities that are suitable for direct infusion into a subject include, but are not limited to: at a dilution and/or concentration suitable for direct infusion; at least 70% viability; wherein the MSCs present in the sample result in at least 20,000 colony forming units (per 1×10⁶ cells); wherein the cells present in the sample comprise at least 90% CD105+ cells.

The methods and systems disclosed herein further enable the sample preparation of MSCs to provide for some or all of at least the above qualities after cryopreservation of the MSCs. The methods and systems disclosed herein further enable the sample preparation of MSCs to provide for some or all of at least the above qualities after thawing cryopreserved MSCs and culturing the MSCs for a period of time. Finally, he methods and systems disclosed herein further enable the sample preparation of MSCs to provide for some or all of at least the above qualities after thawing cryopreserved MSCs, culturing the MSCs for a period of time, and then maintaining the MSCs at a different temperature than the temperature under which the MSCs were cultured post-thaw. In some embodiments, this change in temperature is a cooling. In some embodiments, the MSCs are maintained in hypothermic conditions post-thaw until direct infusion into a subject.

Recovery of MSCs from Processed Bone

In another feature of the systems and methods disclosed herein, a method is provided for preparing a composition of cadaveric human MSCs from bone. In some embodiments, the preparation may include providing a bone derived from a deceased donor, grinding the bone into one or more ground bone segments, filtering the one or more ground bone segments and extracting the cadaveric human MSCs from the one or more ground bone segments. In some embodiments, the MSCs may be recovered from thawed or cryopreserved VB bone fragments. In some embodiments, the extracted cadaveric human MSCs may be adherent vertebral body mesenchymal stem cells (vBA-MSCs). In some embodiments, the extracted cadaveric human MSCs are derived from a bone or fragments thereof that has already been processed to remove bone marrow or derivates thereof associated with the bone or fragment thereof (e.g. bone marrow derived cells, hematopoietic stem cells). In some embodiments, the extracted cadaveric human MSCs are derived from a bone or fragments thereof that has been processed for bone marrow and/or bone marrow-derived cells (e.g. hematopoietic stem cells) as described herein. In some embodiments, the extracted cadaveric human MSCs are derived from the bone grindings and/or segments described herein following filtration and/or extraction and/or isolation of bone marrow and/or bone marrow-derived cells as described herein. The processing and extraction of viable vBA-MSCs from the bone and/or derivates thereof (e.g. bone grindings described herein, bone segments described herein) results in significant improvements in cell yield, especially with respect to total cell yield (vBA-MSCs and hematopoietic stem cells) per weight of bone derived from a donor, and viability of cells with respect to the state of the art. In some embodiments, the vBA-MSCs described herein can be combined with bone marrow-derived MSCs isolated from bone marrow isolated and processed as described herein.

In some embodiments, the extraction of cadaveric human MSCs may include contacting the bone or derivatives thereof with a digestion solution. In some embodiments, the digestion solution may include one or more distinct enzymes. In some embodiments, the one or more distinct enzymes may include one or more collagenases and neutral proteases. In some embodiments, the digestion solution may be present at a ratio of volume to weight of the one or more ground bone segments and enzymatic solution of about 1:1 to about 15:1. In some embodiments, the ratio may be 1:1, 2.5:1, 5:1, 7.5:1, 10:1 and 15:1 (volume:weight). In some embodiments, the combination of one or more collagenases and neutral proteases is used to obtain the highest possible yields of vBA-MSC.

In some embodiments, a collagenase may include Clostridium histolyticum further comprising two active isoforms, C1 and C2. In some embodiments, one or more collagenases comprising isoforms C1 and C2 may be present in the digestion solution at a ratio comprising more collagenase isoform C1 than collagenase isoform C2. In some embodiments, the ratio of collagenase isoform C1 to collagenase isoform C2 may be about 30 to about 70:about 10 to about 29. In some embodiments, the ratio of collagenase isoform C1 to collagenase C2 may be 35:15. In some embodiments, the mass ratio of C1 and C2 for each concentration may be 70:30, 54:46, 37:63, 82:18, 54:46, and 90:10.

In some embodiments, the neutral protease may be Paneibacillus polymyxa neutral protease. In some embodiments, the neutral protease concentration may be about 2 U/ml to about 21 U/ml. In some embodiments, the neutral protease concentration may be about 2 U/ml to about 7 U/ml, about 2 U/ml to about 12 U/ml, about 2 U/ml to about 17 U/ml, about 2 U/ml to about 21 U/ml, about 7 U/ml to about 12 U/ml, about 7 U/ml to about 17 U/ml, about 7 U/ml to about 21 U/ml, about 12 U/ml to about 17 U/ml, about 12 U/ml to about 21 U/ml, or about 17 U/ml to about 21 U/ml. In some embodiments, the neutral protease concentration may be about 2 U/ml, about 7 U/ml, about 12 U/ml, about 17 U/ml, or about 21 U/ml. In some embodiments, the neutral protease concentration may be at least about 2 U/ml, about 7 U/ml, about 12 U/ml, or about 17 U/ml. In some embodiments, the neutral protease concentration may be at most about 7 U/ml, about 12 U/ml, about 17 U/ml, or about 21 U/ml. In some embodiments, the digestion solution may comprise the neutral protease at an activity of about 19.6 U/ml.

In some embodiments, the collagenase concentration is about 0.05 U/ml to about 1.6 U/ml. In some embodiments, the collagenase concentration is about 0.05 U/ml to about 0.1 U/ml, about 0.05 U/ml to about 0.15 U/ml, about 0.05 U/ml to about 0.2 U/ml, about 0.05 U/ml to about 0.25 U/ml, about 0.05 U/ml to about 0.3 U/ml, about 0.05 U/ml to about 0.35 U/ml, about 0.05 U/ml to about 0.4 U/ml, about 0.05 U/ml to about 0.8 U/ml, about 0.05 U/ml to about 1.2 U/ml, about 0.05 U/ml to about 1.6 U/ml, about 0.1 U/ml to about 0.15 U/ml, about 0.1 U/ml to about 0.2 U/ml, about 0.1 U/ml to about 0.25 U/ml, about 0.1 U/ml to about 0.3 U/ml, about 0.1 U/ml to about 0.35 U/ml, about 0.1 U/ml to about 0.4 U/ml, about 0.1 U/ml to about 0.8 U/ml, about 0.1 U/ml to about 1.2 U/ml, about 0.1 U/ml to about 1.6 U/ml, about 0.15 U/ml to about 0.2 U/ml, about 0.15 U/ml to about 0.25 U/ml, about 0.15 U/ml to about 0.3 U/ml, about 0.15 U/ml to about 0.35 U/ml, about 0.15 U/ml to about 0.4 U/ml, about 0.15 U/ml to about 0.8 U/ml, about 0.15 U/ml to about 1.2 U/ml, about 0.15 U/ml to about 1.6 U/ml, about 0.2 U/ml to about 0.25 U/ml, about 0.2 U/ml to about 0.3 U/ml, about 0.2 U/ml to about 0.35 U/ml, about 0.2 U/ml to about 0.4 U/ml, about 0.2 U/ml to about 0.8 U/ml, about 0.2 U/ml to about 1.2 U/ml, about 0.2 U/ml to about 1.6 U/ml, about 0.25 U/ml to about 0.3 U/ml, about 0.25 U/ml to about 0.35 U/ml, about 0.25 U/ml to about 0.4 U/ml, about 0.25 U/ml to about 0.8 U/ml, about 0.25 U/ml to about 1.2 U/ml, about 0.25 U/ml to about 1.6 U/ml, about 0.3 U/ml to about 0.35 U/ml, about 0.3 U/ml to about 0.4 U/ml, about 0.3 U/ml to about 0.8 U/ml, about 0.3 U/ml to about 1.2 U/ml, about 0.3 U/ml to about 1.6 U/ml, about 0.35 U/ml to about 0.4 U/ml, about 0.35 U/ml to about 0.8 U/ml, about 0.35 U/ml to about 1.2 U/ml, about 0.35 U/ml to about 1.6 U/ml, about 0.4 U/ml to about 0.8 U/ml, about 0.4 U/ml to about 1.2 U/ml, about 0.4 U/ml to about 1.6 U/ml, about 0.8 U/ml to about 1.2 U/ml, about 0.8 U/ml to about 1.6 U/ml, or about 1.2 U/ml to about 1.6 U/ml. In some embodiments, the collagenase concentration is about 0.05 U/ml, about 0.1 U/ml, about 0.15 U/ml, about 0.2 U/ml, about 0.25 U/ml, about 0.3 U/ml, about 0.35 U/ml, about 0.4 U/ml, about 0.8 U/ml, about 1.2 U/ml, or about 1.6 U/ml. In some embodiments, the collagenase concentration is at least about 0.05 U/ml, about 0.1 U/ml, about 0.15 U/ml, about 0.2 U/ml, about 0.25 U/ml, about 0.3 U/ml, about 0.35 U/ml, about 0.4 U/ml, about 0.8 U/ml, or about 1.2 U/ml. In some embodiments, the collagenase concentration is at most about 0.1 U/ml, about 0.15 U/ml, about 0.2 U/ml, about 0.25 U/ml, about 0.3 U/ml, about 0.35 U/ml, about 0.4 U/ml, about 0.8 U/ml, about 1.2 U/ml, or about 1.6 U/ml.

In accordance with one aspect of the disclosure, neutral protease concentration and collagenase concentrations (C1 and C2 collagenase) and ratio of solution volume (mis) to bone fragment weight (mgs) are determined.

In some embodiments, the total collagenase concentrations (C1 and C2 collagenase) are about 25 μg/ml to about 100 μg/ml. In some embodiments, the total collagenase concentrations are about 25 μg/ml to about 32.5 μg/ml, about 25 μg/ml to about 47.5 μg/ml, about 25 μg/ml to about 42.5 μg/ml, about 25 μg/ml to about 50 μg/ml, about 25 μg/ml to about 65 μg/ml, about 25 μg/ml to about 77.5 μg/ml, about 25 μg/ml to about 85 μg/ml, about 25 μg/ml to about 100 μg/ml, about 32.5 μg/ml to about 47.5 μg/ml, about 32.5 μg/ml to about 42.5 μg/ml, about 32.5 μg/ml to about 50 μg/ml, about 32.5 μg/ml to about 65 μg/ml, about 32.5 μg/ml to about 77.5 μg/ml, about 32.5 μg/ml to about 85 μg/ml, about 32.5 μg/ml to about 100 μg/ml, about 47.5 μg/ml to about 42.5 μg/ml, about 47.5 μg/ml to about 50 μg/ml, about 47.5 μg/ml to about 65 μg/ml, about 47.5 μg/ml to about 77.5 μg/ml, about 47.5 μg/ml to about 85 μg/ml, about 47.5 μg/ml to about 100 μg/ml, about 42.5 μg/ml to about 50 μg/ml, about 42.5 μg/ml to about 65 μg/ml, about 42.5 μg/ml to about 77.5 μg/ml, about 42.5 μg/ml to about 85 μg/ml, about 42.5 μg/ml to about 100 μg/ml, about 50 μg/ml to about 65 μg/ml, about 50 μg/ml to about 77.5 μg/ml, about 50 μg/ml to about 85 μg/ml, about 50 μg/ml to about 100 μg/ml, about 65 μg/ml to about 77.5 μg/ml, about 65 μg/ml to about 85 μg/ml, about 65 μg/ml to about 100 μg/ml, about 77.5 μg/ml to about 85 μg/ml, about 77.5 μg/ml to about 100 μg/ml, or about 85 μg/ml to about 100 μg/ml. In some embodiments, the total collagenase concentrations are about 25 μg/ml, about 32.5 μg/ml, about 47.5 μg/ml, about 42.5 μg/ml, about 50 μg/ml, about 65 μg/ml, about 77.5 μg/ml, about 85 μg/ml, or about 100 μg/ml. In some embodiments, the total collagenase concentrations are at least about 25 μg/ml, about 32.5 μg/ml, about 47.5 μg/ml, about 42.5 μg/ml, about 50 μg/ml, about 65 μg/ml, about 77.5 μg/ml, or about 85 μg/ml. In some embodiments, the total collagenase concentrations are at most about 32.5 μg/ml, about 47.5 μg/ml, about 42.5 μg/ml, about 50 μg/ml, about 65 μg/ml, about 77.5 μg/ml, about 85 μg/ml, or about 100 μg/ml.

In some embodiments, the mass ratio of C1 and C2 for each concentration are 70:30, 54:46, 37:63, 82:18 and 90:10, respectively.

According to the process, fragments of VB bone are placed in cryoprotectant solution comprised of PLASMA-LYTE™, 2.5% human serum albumin and 10% dimethyl sulfoxide (DMSO) and incubated for 1 hour at 4° C. In some embodiments, the incubation period is about 1 hour to about 3 hours. In some embodiments, the incubation period is about 1 hour to about 1.5 hours, about 1 hour to about 2 hours, about 1 hour to about 2.5 hours, about 1 hour to about 3 hours, about 1.5 hours to about 2 hours, about 1.5 hours to about 2.5 hours, about 1.5 hours to about 3 hours, about 2 hours to about 2.5 hours, about 2 hours to about 3 hours, or about 2.5 hours to about 3 hours. In some embodiments, the incubation period is about 1 hour, about 1.5 hours, about 2 hours, about 2.5 hours, or about 3 hours. In some embodiments, the incubation period is at least about 1 hour, about 1.5 hours, about 2 hours, or about 2.5 hours. In some embodiments, the incubation period is at most about 1.5 hours, about 2 hours, about 2.5 hours, or about 3 hours. The solution is removed and the bone fragments cooled at a rate of −1°/min to −86° C. and then plunged into liquid nitrogen. After 24-48 hours in liquid nitrogen, the bone fragments are thawed rapidly in a water bath set at 37° C. and then washed in saline and digested using the collagenase/protease solution described above.

In some embodiments, the volume-to-weight ratio was 5:1 at an incubation time of 2.5 hours. In some embodiments, the protease produced neutral protease activity of 19.6 U/ml.

The mixture of cells liberated by digesting VB bone fragment is cultured on tissue-coated plastic in the presence of Mesencult medium to select proliferative vBA-MSC. Freshly digested preparations as well as different passages of VB-MSC can be characterized by flow cytometry, colony forming unit-fibroblast (CFU-F) potential, population doubling time (PDT) and trilineage (adipogenic, chondrogenic, and osteogenic) differentiation in vitro.

In some embodiments, the method of cadaveric human MSC extraction disclosed herein may be capable of extracting quantities of about 10 million to about 10 billion. In some embodiments, cadaveric human MSCs may be administered in quantities of about 10 million to about 100 million, about 10 million to about 1 billion, about 10 million to about 10 billion, about 100 million to about 1 billion, about 100 million to about 10 billion, or about 1 billion to about 10 billion. In some embodiments, cadaveric human MSCs may be administered in quantities of about 10 million, about 100 million, about 1 billion, or about 10 billion. In some embodiments, cadaveric human MSCs may be administered in quantities of at least about 10 million, about 100 million, or about 1 billion. In some embodiments, cadaveric human MSCs may be administered in quantities of at most about 100 million, about 1 billion, or about 10 billion.

### MSC Compositions

It has been shown that MSC are a potentially useful adjuvant to stem cell transplants (SCT) for promoting mixed chimerisms as well as promoting complementary peripheral immunomodulatory functions. It has been demonstrated the therapeutic potential of MSC for inducing operational tolerance of SOT and VCA, providing proof-of-principle for clinical testing in the transplant setting. The effect of MSC infusion, including in humans and nonhuman primates, is to skew the T cell population in favor of TREG over TEM cells. It is evident that mixed chimerisim is essential for central tolerance of SOT and VCA, which is complemented by expansion of peripheral alloantigen-specific regulatory T cells (TREGs) and donor-derived tolerogenic dendritic cells (TolDC).

In some embodiments, cadaveric MSCs may administered prophylactically, perioperatively or postoperatively with SOT or other VCA procedures. In some embodiments, cadaveric MSCs administered may comprise cadaveric human MSCs derived from bone marrow, adherent vertebral body MSCs (vBA-MSCs), or both.

In some embodiments, the composition of cadaveric human MSCs provided for inhibiting an immune response may be administered in quantities of about 10 million to about 10 billion. In some embodiments, cadaveric human MSCs may be administered in quantities of about 10 million to about 100 million, about 10 million to about 1 billion, about 10 million to about 10 billion, about 100 million to about 1 billion, about 100 million to about 10 billion, or about 1 billion to about 10 billion. In some embodiments, cadaveric human MSCs may be administered in quantities of about 10 million, about 100 million, about 1 billion, or about 10 billion. In some embodiments, cadaveric human MSCs may be administered in quantities of at least about 10 million, about 100 million, or about 1 billion. In some embodiments, cadaveric human MSCs may be administered in quantities of at most about 100 million, about 1 billion, or about 10 billion.

In some embodiments, the composition of cadaveric MSCs (e.g. vBA-MSCs) may be comprised of less than 5% CD45+. In some embodiments, the composition of cadaveric MSCs may be comprised of less than about 0.5% CD45+ to about 10% CD45+. In some embodiments, the composition of cadaveric MSCs may be comprised of less than about 10% CD45+ to about 9% CD45+, about 10% CD45+ to about 8% CD45+, about 10% CD45+ to about 7% CD45+, about 10% CD45+ to about 6% CD45+, about 10% CD45+ to about 5% CD45+, about 10% CD45+ to about 4% CD45+, about 10% CD45+ to about 3% CD45+, about 10% CD45+ to about 2% CD45+, about 10% CD45+ to about 1% CD45+, about 10% CD45+ to about 0.5% CD45+, about 9% CD45+ to about 8% CD45+, about 9% CD45+ to about 7% CD45+, about 9% CD45+ to about 6% CD45+, about 9% CD45+ to about 5% CD45+, about 9% CD45+ to about 4% CD45+, about 9% CD45+ to about 3% CD45+, about 9% CD45+ to about 2% CD45+, about 9% CD45+ to about 1% CD45+, about 9% CD45+ to about 0.5% CD45+, about 8% CD45+ to about 7% CD45+, about 8% CD45+ to about 6% CD45+, about 8% CD45+ to about 5% CD45+, about 8% CD45+ to about 4% CD45+, about 8% CD45+ to about 3% CD45+, about 8% CD45+ to about 2% CD45+, about 8% CD45+ to about 1% CD45+, about 8% CD45+ to about 0.5% CD45+, about 7% CD45+ to about 6% CD45+, about 7% CD45+ to about 5% CD45+, about 7% CD45+ to about 4% CD45+, about 7% CD45+ to about 3% CD45+, about 7% CD45+ to about 2% CD45+, about 7% CD45+ to about 1% CD45+, about 7% CD45+ to about 0.5% CD45+, about 6% CD45+ to about 5% CD45+, about 6% CD45+ to about 4% CD45+, about 6% CD45+ to about 3% CD45+, about 6% CD45+ to about 2% CD45+, about 6% CD45+ to about 1% CD45+, about 6% CD45+ to about 0.5% CD45+, about 5% CD45+ to about 4% CD45+, about 5% CD45+ to about 3% CD45+, about 5% CD45+ to about 2% CD45+, about 5% CD45+ to about 1% CD45+, about 5% CD45+ to about 0.5% CD45+, about 4% CD45+ to about 3% CD45+, about 4% CD45+ to about 2% CD45+, about 4% CD45+ to about 1% CD45+, about 4% CD45+ to about 0.5% CD45+, about 3% CD45+ to about 2% CD45+, about 3% CD45+ to about 1% CD45+, about 3% CD45+ to about 0.5% CD45+, about 2% CD45+ to about 1% CD45+, about 2% CD45+ to about 0.5% CD45+, or about 1% CD45+ to about 0.5% CD45+. In some embodiments, the composition of cadaveric MSCs may be comprised of less than about 10% CD45+, about 9% CD45+, about 8% CD45+, about 7% CD45+, about 6% CD45+, about 5% CD45+, about 4% CD45+, about 3% CD45+, about 2% CD45+, about 1% CD45+, or about 0.5% CD45+. In some embodiments, the composition of cadaveric MSCs may be comprised of less than at least about 10% CD45+, about 9% CD45+, about 8% CD45+, about 7% CD45+, about 6% CD45+, about 5% CD45+, about 4% CD45+, about 3% CD45+, about 2% CD45+, or about 1% CD45+. In some embodiments, the composition of cadaveric MSCs may be comprised of less than at most about 9% CD45+, about 8% CD45+, about 7% CD45+, about 6% CD45+, about 5% CD45+, about 4% CD45+, about 3% CD45+, about 2% CD45+, about 1% CD45+, or about 0.5% CD45+.

In some embodiments, the composition of cadaveric MSCs (e.g. vBA-MSCs) may comprise more than 1% CD45+ cells. In some embodiments, the composition of cadaveric MSCs may comprise more than 1.1% CD45+ cells. In some embodiments, the composition of cadaveric MSCs may comprise more than 1.2% CD45+ cells. In some embodiments, the composition of cadaveric MSCs may comprise more than 1.3% CD45+ cells. In some embodiments, the composition of cadaveric MSCs may comprise more than 1.4% CD45+ cells. In some embodiments, the composition of cadaveric MSCs may comprise more than 1.5% CD45+ cells. In some embodiments, the composition of cadaveric MSCs may comprise more than 1.6% CD45+ cells. In some embodiments, the composition of cadaveric MSCs may comprise more than 1.7% CD45+ cells. In some embodiments, the composition of cadaveric MSCs may comprise more than 1.8% CD45+ cells. In some embodiments, the composition of cadaveric MSCs may comprise more than 1.9% CD45+ cells. In some embodiments, the composition of cadaveric MSCs may comprise more than 2% CD45+ cells.

In some embodiments, the composition of cadaveric MSCs may be comprised of at least 90% CD105+ cells. In some embodiments, the composition of cadaveric MSCs may be comprised of at least about 70% CD105+ cells to about 100% CD105+ cells. In some embodiments, the composition of cadaveric MSCs may be comprised of at least about 100% CD105+ cells to about 95% CD105+ cells, about 100% CD105+ cells to about 94% CD105+ cells, about 100% CD105+ cells to about 93% CD105+ cells, about 100% CD105+ cells to about 92% CD105+ cells, about 100% CD105+ cells to about 91% CD105+ cells, about 100% CD105+ cells to about 90% CD105+ cells, about 100% CD105+ cells to about 85% CD105+ cells, about 100% CD105+ cells to about 80% CD105+ cells, about 100% CD105+ cells to about 75% CD105+ cells, about 100% CD105+ cells to about 70% CD105+ cells, about 95% CD105+ cells to about 94% CD105+ cells, about 95% CD105+ cells to about 93% CD105+ cells, about 95% CD105+ cells to about 92% CD105+ cells, about 95% CD105+ cells to about 91% CD105+ cells, about 95% CD105+ cells to about 90% CD105+ cells, about 95% CD105+ cells to about 85% CD105+ cells, about 95% CD105+ cells to about 80% CD105+ cells, about 95% CD105+ cells to about 75% CD105+ cells, about 95% CD105+ cells to about 70% CD105+ cells, about 94% CD105+ cells to about 93% CD105+ cells, about 94% CD105+ cells to about 92% CD105+ cells, about 94% CD105+ cells to about 91% CD105+ cells, about 94% CD105+ cells to about 90% CD105+ cells, about 94% CD105+ cells to about 85% CD105+ cells, about 94% CD105+ cells to about 80% CD105+ cells, about 94% CD105+ cells to about 75% CD105+ cells, about 94% CD105+ cells to about 70% CD105+ cells, about 93% CD105+ cells to about 92% CD105+ cells, about 93% CD105+ cells to about 91% CD105+ cells, about 93% CD105+ cells to about 90% CD105+ cells, about 93% CD105+ cells to about 85% CD105+ cells, about 93% CD105+ cells to about 80% CD105+ cells, about 93% CD105+ cells to about 75% CD105+ cells, about 93% CD105+ cells to about 70% CD105+ cells, about 92% CD105+ cells to about 91% CD105+ cells, about 92% CD105+ cells to about 90% CD105+ cells, about 92% CD105+ cells to about 85% CD105+ cells, about 92% CD105+ cells to about 80% CD105+ cells, about 92% CD105+ cells to about 75% CD105+ cells, about 92% CD105+ cells to about 70% CD105+ cells, about 91% CD105+ cells to about 90% CD105+ cells, about 91% CD105+ cells to about 85% CD105+ cells, about 91% CD105+ cells to about 80% CD105+ cells, about 91% CD105+ cells to about 75% CD105+ cells, about 91% CD105+ cells to about 70% CD105+ cells, about 90% CD105+ cells to about 85% CD105+ cells, about 90% CD105+ cells to about 80% CD105+ cells, about 90% CD105+ cells to about 75% CD105+ cells, about 90% CD105+ cells to about 70% CD105+ cells, about 85% CD105+ cells to about 80% CD105+ cells, about 85% CD105+ cells to about 75% CD105+ cells, about 85% CD105+ cells to about 70% CD105+ cells, about 80% CD105+ cells to about 75% CD105+ cells, about 80% CD105+ cells to about 70% CD105+ cells, or about 75% CD105+ cells to about 70% CD105+ cells. In some embodiments, the composition of cadaveric MSCs may be comprised of at least about 100% CD105+ cells, about 95% CD105+ cells, about 94% CD105+ cells, about 93% CD105+ cells, about 92% CD105+ cells, about 91% CD105+ cells, about 90% CD105+ cells, about 85% CD105+ cells, about 80% CD105+ cells, about 75% CD105+ cells, or about 70% CD105+ cells. In some embodiments, the composition of cadaveric MSCs may be comprised of at least at least about 100% CD105+ cells, about 95% CD105+ cells, about 94% CD105+ cells, about 93% CD105+ cells, about 92% CD105+ cells, about 91% CD105+ cells, about 90% CD105+ cells, about 85% CD105+ cells, about 80% CD105+ cells, or about 75% CD105+ cells. In some embodiments, the composition of cadaveric MSCs may be comprised of at least at most about 95% CD105+ cells, about 94% CD105+ cells, about 93% CD105+ cells, about 92% CD105+ cells, about 91% CD105+ cells, about 90% CD105+ cells, about 85% CD105+ cells, about 80% CD105+ cells, about 75% CD105+ cells, or about 70% CD105+ cells.

In some embodiments, the composition of cadaveric MSCs may be comprised of at least 90% CD166+ cells. In some embodiments, the composition of cadaveric MSCs may be comprised of at least about 70% CD166+ cells to about 100% CD166+ cells. In some embodiments, the composition of cadaveric MSCs may be comprised of at least about 100% CD166+ cells to about 95% CD166+ cells, about 100% CD166+ cells to about 94% CD166+ cells, about 100% CD166+ cells to about 93% CD166+ cells, about 100% CD166+ cells to about 92% CD166+ cells, about 100% CD166+ cells to about 91% CD166+ cells, about 100% CD166+ cells to about 90% CD166+ cells, about 100% CD166+ cells to about 85% CD166+ cells, about 100% CD166+ cells to about 80% CD166+ cells, about 100% CD166+ cells to about 75% CD166+ cells, about 100% CD166+ cells to about 70% CD166+ cells, about 95% CD166+ cells to about 94% CD166+ cells, about 95% CD166+ cells to about 93% CD166+ cells, about 95% CD166+ cells to about 92% CD166+ cells, about 95% CD166+ cells to about 91% CD166+ cells, about 95% CD166+ cells to about 90% CD166+ cells, about 95% CD166+ cells to about 85% CD166+ cells, about 95% CD166+ cells to about 80% CD166+ cells, about 95% CD166+ cells to about 75% CD166+ cells, about 95% CD166+ cells to about 70% CD166+ cells, about 94% CD166+ cells to about 93% CD166+ cells, about 94% CD166+ cells to about 92% CD166+ cells, about 94% CD166+ cells to about 91% CD166+ cells, about 94% CD166+ cells to about 90% CD166+ cells, about 94% CD166+ cells to about 85% CD166+ cells, about 94% CD166+ cells to about 80% CD166+ cells, about 94% CD166+ cells to about 75% CD166+ cells, about 94% CD166+ cells to about 70% CD166+ cells, about 93% CD166+ cells to about 92% CD166+ cells, about 93% CD166+ cells to about 91% CD166+ cells, about 93% CD166+ cells to about 90% CD166+ cells, about 93% CD166+ cells to about 85% CD166+ cells, about 93% CD166+ cells to about 80% CD166+ cells, about 93% CD166+ cells to about 75% CD166+ cells, about 93% CD166+ cells to about 70% CD166+ cells, about 92% CD166+ cells to about 91% CD166+ cells, about 92% CD166+ cells to about 90% CD166+ cells, about 92% CD166+ cells to about 85% CD166+ cells, about 92% CD166+ cells to about 80% CD166+ cells, about 92% CD166+ cells to about 75% CD166+ cells, about 92% CD166+ cells to about 70% CD166+ cells, about 91% CD166+ cells to about 90% CD166+ cells, about 91% CD166+ cells to about 85% CD166+ cells, about 91% CD166+ cells to about 80% CD166+ cells, about 91% CD166+ cells to about 75% CD166+ cells, about 91% CD166+ cells to about 70% CD166+ cells, about 90% CD166+ cells to about 85% CD166+ cells, about 90% CD166+ cells to about 80% CD166+ cells, about 90% CD166+ cells to about 75% CD166+ cells, about 90% CD166+ cells to about 70% CD166+ cells, about 85% CD166+ cells to about 80% CD166+ cells, about 85% CD166+ cells to about 75% CD166+ cells, about 85% CD166+ cells to about 70% CD166+ cells, about 80% CD166+ cells to about 75% CD166+ cells, about 80% CD166+ cells to about 70% CD166+ cells, or about 75% CD166+ cells to about 70% CD166+ cells. In some embodiments, the composition of cadaveric MSCs may be comprised of at least about 100% CD166+ cells, about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, about 75% CD166+ cells, or about 70% CD166+ cells. In some embodiments, the composition of cadaveric MSCs may be comprised of at least at least about 100% CD166+ cells, about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, or about 75% CD166+ cells. In some embodiments, the composition of cadaveric MSCs may be comprised of at least at most about 95% CD166+ cells, about 94% CD166+ cells, about 93% CD166+ cells, about 92% CD166+ cells, about 91% CD166+ cells, about 90% CD166+ cells, about 85% CD166+ cells, about 80% CD166+ cells, about 75% CD166+ cells, or about 70% CD166+ cells.

### Culturing of MSCs

In one aspect of the present disclosure, extracted MSCs may be (e.g. vBA-MSCs) cultured and passaged to realize clinical scale MSC preparation having a desired number of MSCs with the antigen profiles taught herein. In some embodiments, a clinical scale preparation may be obtained by serial passage expansion where each passage includes a step of splitting the previous culture into a plurality of cultures at a given ratio. Each passaging step increases the number of concurrent cultures in the preparation. In some embodiments, clinical scale preparations having the instant preparation profiles, e.g. antigen profile, TNFRI profile, cryopreservation profile, differentiation profile, and/or sterility (with respect to pathogens) are successfully produced.

In some embodiments, extracted MSCs are cultured in a medium wherein the medium is configured to generate MSCs having the instant preparation profiles, e.g. antigen profile, TNFRI profile, cryopreservation profile, differentiation profile, and/or sterility (with respect to pathogens). In some embodiments, the medium comprises minimal essential medium (MEM). In some embodiments, the medium comprises alpha MEM. In some embodiments, the medium comprises human platelet lysate (hPL). In some embodiments, the medium comprises carrier free fibroblast growth factor (FGF). In some embodiments, the medium comprises carrier free epidermal growth factor (EGF). In some embodiments, the medium comprises alpha MEM, hPL, FGF, EGF, or any combination thereof. In some embodiments, the medium comprises alpha MEM, hPL, FGF, and EGF. In some embodiments, the medium does not further require heparin.

In some embodiments, hPL is present in the medium at about 1% to about 21%. In some embodiments, hPL is present in the medium at about 1% to about 3%, about 1% to about 5%, about 1% to about 7%, about 1% to about 9%, about 1% to about 10%, about 1% to about 11%, about 1% to about 13%, about 1% to about 15%, about 1% to about 17%, about 1% to about 19%, about 1% to about 21%, about 3% to about 5%, about 3% to about 7%, about 3% to about 9%, about 3% to about 10%, about 3% to about 11%, about 3% to about 13%, about 3% to about 15%, about 3% to about 17%, about 3% to about 19%, about 3% to about 21%, about 5% to about 7%, about 5% to about 9%, about 5% to about 10%, about 5% to about 11%, about 5% to about 13%, about 5% to about 15%, about 5% to about 17%, about 5% to about 19%, about 5% to about 21%, about 7% to about 9%, about 7% to about 10%, about 7% to about 11%, about 7% to about 13%, about 7% to about 15%, about 7% to about 17%, about 7% to about 19%, about 7% to about 21%, about 9% to about 10%, about 9% to about 11%, about 9% to about 13%, about 9% to about 15%, about 9% to about 17%, about 9% to about 19%, about 9% to about 21%, about 10% to about 11%, about 10% to about 13%, about 10% to about 15%, about 10% to about 17%, about 10% to about 19%, about 10% to about 21%, about 11% to about 13%, about 11% to about 15%, about 11% to about 17%, about 11% to about 19%, about 11% to about 21%, about 13% to about 15%, about 13% to about 17%, about 13% to about 19%, about 13% to about 21%, about 15% to about 17%, about 15% to about 19%, about 15% to about 21%, about 17% to about 19%, about 17% to about 21%, or about 19% to about 21%. In some embodiments, hPL is present in the medium at about 1%, about 3%, about 5%, about 7%, about 9%, about 10%, about 11%, about 13%, about 15%, about 17%, about 19%, or about 21%. In some embodiments, hPL is present in the medium at least about 1%, about 3%, about 5%, about 7%, about 9%, about 10%, about 11%, about 13%, about 15%, about 17%, or about 19%. In some embodiments, hPL is present in the medium at most about 3%, about 5%, about 7%, about 9%, about 10%, about 11%, about 13%, about 15%, about 17%, about 19%, or about 21%. In some embodiments, FGF is present in the medium at about 0.5 ng/mL to about 5 ng/mL. In some embodiments, FGF is present in the medium at about 0.5 ng/mL to about 1 ng/mL, about 0.5 ng/mL to about 1.5 ng/mL, about 0.5 ng/mL to about 2 ng/mL, about 0.5 ng/mL to about 2.5 ng/mL, about 0.5 ng/mL to about 3 ng/mL, about 0.5 ng/mL to about 3.5 ng/mL, about 0.5 ng/mL to about 4 ng/mL, about 0.5 ng/mL to about 4.5 ng/mL, about 0.5 ng/mL to about 5 ng/mL, about 1 ng/mL to about 1.5 ng/mL, about 1 ng/mL to about 2 ng/mL, about 1 ng/mL to about 2.5 ng/mL, about 1 ng/mL to about 3 ng/mL, about 1 ng/mL to about 3.5 ng/mL, about 1 ng/mL to about 4 ng/mL, about 1 ng/mL to about 4.5 ng/mL, about 1 ng/mL to about 5 ng/mL, about 1.5 ng/mL to about 2 ng/mL, about 1.5 ng/mL to about 2.5 ng/mL, about 1.5 ng/mL to about 3 ng/mL, about 1.5 ng/mL to about 3.5 ng/mL, about 1.5 ng/mL to about 4 ng/mL, about 1.5 ng/mL to about 4.5 ng/mL, about 1.5 ng/mL to about 5 ng/mL, about 2 ng/mL to about 2.5 ng/mL, about 2 ng/mL to about 3 ng/mL, about 2 ng/mL to about 3.5 ng/mL, about 2 ng/mL to about 4 ng/mL, about 2 ng/mL to about 4.5 ng/mL, about 2 ng/mL to about 5 ng/mL, about 2.5 ng/mL to about 3 ng/mL, about 2.5 ng/mL to about 3.5 ng/mL, about 2.5 ng/mL to about 4 ng/mL, about 2.5 ng/mL to about 4.5 ng/mL, about 2.5 ng/mL to about 5 ng/mL, about 3 ng/mL to about 3.5 ng/mL, about 3 ng/mL to about 4 ng/mL, about 3 ng/mL to about 4.5 ng/mL, about 3 ng/mL to about 5 ng/mL, about 3.5 ng/mL to about 4 ng/mL, about 3.5 ng/mL to about 4.5 ng/mL, about 3.5 ng/mL to about 5 ng/mL, about 4 ng/mL to about 4.5 ng/mL, about 4 ng/mL to about 5 ng/mL, or about 4.5 ng/mL to about 5 ng/mL. In some embodiments, FGF is present in the medium at about 0.5 ng/mL, about 1 ng/mL, about 1.5 ng/mL, about 2 ng/mL, about 2.5 ng/mL, about 3 ng/mL, about 3.5 ng/mL, about 4 ng/mL, about 4.5 ng/mL, or about 5 ng/mL. In some embodiments, FGF is present in the medium at least about 0.5 ng/mL, about 1 ng/mL, about 1.5 ng/mL, about 2 ng/mL, about 2.5 ng/mL, about 3 ng/mL, about 3.5 ng/mL, about 4 ng/mL, or about 4.5 ng/mL. In some embodiments, FGF is present in the medium at most about 1 ng/mL, about 1.5 ng/mL, about 2 ng/mL, about 2.5 ng/mL, about 3 ng/mL, about 3.5 ng/mL, about 4 ng/mL, about 4.5 ng/mL, or about 5 ng/mL.

In some embodiments, EGF is present in the medium at about 0.5 ng/mL to about 5 ng/mL. In some embodiments, EGF is present in the medium at about 0.5 ng/mL to about 1 ng/mL, about 0.5 ng/mL to about 1.5 ng/mL, about 0.5 ng/mL to about 2 ng/mL, about 0.5 ng/mL to about 2.5 ng/mL, about 0.5 ng/mL to about 3 ng/mL, about 0.5 ng/mL to about 3.5 ng/mL, about 0.5 ng/mL to about 4 ng/mL, about 0.5 ng/mL to about 4.5 ng/mL, about 0.5 ng/mL to about 5 ng/mL, about 1 ng/mL to about 1.5 ng/mL, about 1 ng/mL to about 2 ng/mL, about 1 ng/mL to about 2.5 ng/mL, about 1 ng/mL to about 3 ng/mL, about 1 ng/mL to about 3.5 ng/mL, about 1 ng/mL to about 4 ng/mL, about 1 ng/mL to about 4.5 ng/mL, about 1 ng/mL to about 5 ng/mL, about 1.5 ng/mL to about 2 ng/mL, about 1.5 ng/mL to about 2.5 ng/mL, about 1.5 ng/mL to about 3 ng/mL, about 1.5 ng/mL to about 3.5 ng/mL, about 1.5 ng/mL to about 4 ng/mL, about 1.5 ng/mL to about 4.5 ng/mL, about 1.5 ng/mL to about 5 ng/mL, about 2 ng/mL to about 2.5 ng/mL, about 2 ng/mL to about 3 ng/mL, about 2 ng/mL to about 3.5 ng/mL, about 2 ng/mL to about 4 ng/mL, about 2 ng/mL to about 4.5 ng/mL, about 2 ng/mL to about 5 ng/mL, about 2.5 ng/mL to about 3 ng/mL, about 2.5 ng/mL to about 3.5 ng/mL, about 2.5 ng/mL to about 4 ng/mL, about 2.5 ng/mL to about 4.5 ng/mL, about 2.5 ng/mL to about 5 ng/mL, about 3 ng/mL to about 3.5 ng/mL, about 3 ng/mL to about 4 ng/mL, about 3 ng/mL to about 4.5 ng/mL, about 3 ng/mL to about 5 ng/mL, about 3.5 ng/mL to about 4 ng/mL, about 3.5 ng/mL to about 4.5 ng/mL, about 3.5 ng/mL to about 5 ng/mL, about 4 ng/mL to about 4.5 ng/mL, about 4 ng/mL to about 5 ng/mL, or about 4.5 ng/mL to about 5 ng/mL. In some embodiments, EGF is present in the medium at about 0.5 ng/mL, about 1 ng/mL, about 1.5 ng/mL, about 2 ng/mL, about 2.5 ng/mL, about 3 ng/mL, about 3.5 ng/mL, about 4 ng/mL, about 4.5 ng/mL, or about 5 ng/mL. In some embodiments, EGF is present in the medium at least about 0.5 ng/mL, about 1 ng/mL, about 1.5 ng/mL, about 2 ng/mL, about 2.5 ng/mL, about 3 ng/mL, about 3.5 ng/mL, about 4 ng/mL, or about 4.5 ng/mL. In some embodiments, EGF is present in the medium at most about 1 ng/mL, about 1.5 ng/mL, about 2 ng/mL, about 2.5 ng/mL, about 3 ng/mL, about 3.5 ng/mL, about 4 ng/mL, about 4.5 ng/mL, or about 5 ng/mL.

In some embodiments, the medium comprises a modified alpha MEM. In some embodiments, the modified alpha MEM comprises one or more inorganic salts, one or more amino acids, one or more vitamins, glucose, lipoic acid, sodium bicarbonate, sodium pyruvate, or any combination thereof.

In some embodiments, the one or more inorganic salts comprise calcium chloride (dihydrate), magnesium sulfate (heptahydrate), potassium chloride, sodium chloride, sodium phosphate monobasic (dehydrate), or any combination thereof. In some embodiments, each inorganic salt present in the medium is present at about 100 mg/Liter to about 800 mg/Liter. In some embodiments, each inorganic salt present in the medium is present at about 100 mg/Liter to about 200 mg/Liter, about 100 mg/Liter to about 300 mg/Liter, about 100 mg/Liter to about 400 mg/Liter, about 100 mg/Liter to about 500 mg/Liter, about 100 mg/Liter to about 600 mg/Liter, about 100 mg/Liter to about 700 mg/Liter, about 100 mg/Liter to about 800 mg/Liter, about 200 mg/Liter to about 300 mg/Liter, about 200 mg/Liter to about 400 mg/Liter, about 200 mg/Liter to about 500 mg/Liter, about 200 mg/Liter to about 600 mg/Liter, about 200 mg/Liter to about 700 mg/Liter, about 200 mg/Liter to about 800 mg/Liter, about 300 mg/Liter to about 400 mg/Liter, about 300 mg/Liter to about 500 mg/Liter, about 300 mg/Liter to about 600 mg/Liter, about 300 mg/Liter to about 700 mg/Liter, about 300 mg/Liter to about 800 mg/Liter, about 400 mg/Liter to about 500 mg/Liter, about 400 mg/Liter to about 600 mg/Liter, about 400 mg/Liter to about 700 mg/Liter, about 400 mg/Liter to about 800 mg/Liter, about 500 mg/Liter to about 600 mg/Liter, about 500 mg/Liter to about 700 mg/Liter, about 500 mg/Liter to about 800 mg/Liter, about 600 mg/Liter to about 700 mg/Liter, about 600 mg/Liter to about 800 mg/Liter, or about 700 mg/Liter to about 800 mg/Liter. In some embodiments, each inorganic salt present in the medium is present at about 100 mg/Liter, about 200 mg/Liter, about 300 mg/Liter, about 400 mg/Liter, about 500 mg/Liter, about 600 mg/Liter, about 700 mg/Liter, or about 800 mg/Liter. In some embodiments, each inorganic salt present in the medium is present at least about 100 mg/Liter, about 200 mg/Liter, about 300 mg/Liter, about 400 mg/Liter, about 500 mg/Liter, about 600 mg/Liter, or about 700 mg/Liter. In some embodiments, each inorganic salt present in the medium is present at most about 200 mg/Liter, about 300 mg/Liter, about 400 mg/Liter, about 500 mg/Liter, about 600 mg/Liter, about 700 mg/Liter, or about 800 mg/Liter.

In some embodiments, the one or more amino acids comprise glycine, alanine, alanyl-glutamine, arginine (HCl), asparagine (monohydrate), aspartic acid, cysteine (HCl) (monohydrate), cystine, glutamic acid, histidine, isoleucine, leucine, lysine, methionine, phenylalanine, proline, serine, threonine, tryptophan, tyrosine, valine, or any combination thereof. In some embodiments, the one or more amino acids are present in the L isoform. In some embodiments, the one or more amino acids are present in the D isoform. In some embodiments, the one or more amino acids are present in both isoforms. In some embodiments, each amino acid present in the medium is present at about 10 mg/Liter to about 100 mg/Liter. In some embodiments, each amino acid present in the medium is present at about 10 mg/Liter to about 20 mg/Liter, about 10 mg/Liter to about 30 mg/Liter, about 10 mg/Liter to about 40 mg/Liter, about 10 mg/Liter to about 50 mg/Liter, about 10 mg/Liter to about 60 mg/Liter, about 10 mg/Liter to about 70 mg/Liter, about 10 mg/Liter to about 80 mg/Liter, about 10 mg/Liter to about 90 mg/Liter, about 10 mg/Liter to about 100 mg/Liter, about 20 mg/Liter to about 30 mg/Liter, about 20 mg/Liter to about 40 mg/Liter, about 20 mg/Liter to about 50 mg/Liter, about 20 mg/Liter to about 60 mg/Liter, about 20 mg/Liter to about 70 mg/Liter, about 20 mg/Liter to about 80 mg/Liter, about 20 mg/Liter to about 90 mg/Liter, about 20 mg/Liter to about 100 mg/Liter, about 30 mg/Liter to about 40 mg/Liter, about 30 mg/Liter to about 50 mg/Liter, about 30 mg/Liter to about 60 mg/Liter, about 30 mg/Liter to about 70 mg/Liter, about 30 mg/Liter to about 80 mg/Liter, about 30 mg/Liter to about 90 mg/Liter, about 30 mg/Liter to about 100 mg/Liter, about 40 mg/Liter to about 50 mg/Liter, about 40 mg/Liter to about 60 mg/Liter, about 40 mg/Liter to about 70 mg/Liter, about 40 mg/Liter to about 80 mg/Liter, about 40 mg/Liter to about 90 mg/Liter, about 40 mg/Liter to about 100 mg/Liter, about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 70 mg/Liter to about 80 mg/Liter, about 70 mg/Liter to about 90 mg/Liter, about 70 mg/Liter to about 100 mg/Liter, about 80 mg/Liter to about 90 mg/Liter, about 80 mg/Liter to about 100 mg/Liter, or about 90 mg/Liter to about 100 mg/Liter. In some embodiments, each amino acid present in the medium is present at about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, or about 100 mg/Liter. In some embodiments, each amino acid present in the medium is present at least about 10 mg/Liter, about 20 mg/Liter, about 30 mg/Liter, about 40 mg/Liter, about 50 mg/Liter, about 60 mg/Liter, about 70 mg/Liter, about 80 mg/Liter, or about 90 mg/Liter. In some embodiments, each amino acid present in the medium is present at most about 20 mg/Liter, about 30 mg/Liter, about 40 mg/Liter, about 50 mg/Liter, about 60 mg/Liter, about 70 mg/Liter, about 80 mg/Liter, about 90 mg/Liter, or about 100 mg/Liter. In some embodiments, each amino acid present in the medium is present at about 100 mg/Liter to about 500 mg/Liter. In some embodiments, each amino acid present in the medium is present at about 100 mg/Liter to about 200 mg/Liter, about 100 mg/Liter to about 300 mg/Liter, about 100 mg/Liter to about 400 mg/Liter, about 100 mg/Liter to about 500 mg/Liter, about 200 mg/Liter to about 300 mg/Liter, about 200 mg/Liter to about 400 mg/Liter, about 200 mg/Liter to about 500 mg/Liter, about 300 mg/Liter to about 400 mg/Liter, about 300 mg/Liter to about 500 mg/Liter, or about 400 mg/Liter to about 500 mg/Liter. In some embodiments, each amino acid present in the medium is present at about 100 mg/Liter, about 200 mg/Liter, about 300 mg/Liter, about 400 mg/Liter, or about 500 mg/Liter. In some embodiments, each amino acid present in the medium is present at least about 100 mg/Liter, about 200 mg/Liter, about 300 mg/Liter, or about 400 mg/Liter. In some embodiments, each amino acid present in the medium is present at most about 200 mg/Liter, about 300 mg/Liter, about 400 mg/Liter, or about 500 mg/Liter.

In some embodiments, the one or more vitamins comprise ascorbic acid, biotin, choline chloride, calcium pantothenate, folic acid, myo-inositol, niacinamide, pyridoxal (HCl), pyruvic acid (sodium salt), riboflavin, thiamine (HCl), vitamin B12, or any combination thereof. In some embodiments, the one or more vitamins are present in the L isoform. In some embodiments, the one or more vitamins are present in the D isoform. In some embodiments, the one or more vitamins are present in both isoforms. In some embodiments, each vitamin present in the medium is present at about 0.1 mg/Liter to about 2 mg/Liter. In some embodiments, each vitamin present in the medium is present at about 0.1 mg/Liter to about 0.3 mg/Liter, about 0.1 mg/Liter to about 0.5 mg/Liter, about 0.1 mg/Liter to about 0.7 mg/Liter, about 0.1 mg/Liter to about 0.9 mg/Liter, about 0.1 mg/Liter to about 1.1 mg/Liter, about 0.1 mg/Liter to about 1.3 mg/Liter, about 0.1 mg/Liter to about 1.5 mg/Liter, about 0.1 mg/Liter to about 1.7 mg/Liter, about 0.1 mg/Liter to about 1.9 mg/Liter, about 0.1 mg/Liter to about 2 mg/Liter, about 0.3 mg/Liter to about 0.5 mg/Liter, about 0.3 mg/Liter to about 0.7 mg/Liter, about 0.3 mg/Liter to about 0.9 mg/Liter, about 0.3 mg/Liter to about 1.1 mg/Liter, about 0.3 mg/Liter to about 1.3 mg/Liter, about 0.3 mg/Liter to about 1.5 mg/Liter, about 0.3 mg/Liter to about 1.7 mg/Liter, about 0.3 mg/Liter to about 1.9 mg/Liter, about 0.3 mg/Liter to about 2 mg/Liter, about 0.5 mg/Liter to about 0.7 mg/Liter, about 0.5 mg/Liter to about 0.9 mg/Liter, about 0.5 mg/Liter to about 1.1 mg/Liter, about 0.5 mg/Liter to about 1.3 mg/Liter, about 0.5 mg/Liter to about 1.5 mg/Liter, about 0.5 mg/Liter to about 1.7 mg/Liter, about 0.5 mg/Liter to about 1.9 mg/Liter, about 0.5 mg/Liter to about 2 mg/Liter, about 0.7 mg/Liter to about 0.9 mg/Liter, about 0.7 mg/Liter to about 1.1 mg/Liter, about 0.7 mg/Liter to about 1.3 mg/Liter, about 0.7 mg/Liter to about 1.5 mg/Liter, about 0.7 mg/Liter to about 1.7 mg/Liter, about 0.7 mg/Liter to about 1.9 mg/Liter, about 0.7 mg/Liter to about 2 mg/Liter, about 0.9 mg/Liter to about 1.1 mg/Liter, about 0.9 mg/Liter to about 1.3 mg/Liter, about 0.9 mg/Liter to about 1.5 mg/Liter, about 0.9 mg/Liter to about 1.7 mg/Liter, about 0.9 mg/Liter to about 1.9 mg/Liter, about 0.9 mg/Liter to about 2 mg/Liter, about 1.1 mg/Liter to about 1.3 mg/Liter, about 1.1 mg/Liter to about 1.5 mg/Liter, about 1.1 mg/Liter to about 1.7 mg/Liter, about 1.1 mg/Liter to about 1.9 mg/Liter, about 1.1 mg/Liter to about 2 mg/Liter, about 1.3 mg/Liter to about 1.5 mg/Liter, about 1.3 mg/Liter to about 1.7 mg/Liter, about 1.3 mg/Liter to about 1.9 mg/Liter, about 1.3 mg/Liter to about 2 mg/Liter, about 1.5 mg/Liter to about 1.7 mg/Liter, about 1.5 mg/Liter to about 1.9 mg/Liter, about 1.5 mg/Liter to about 2 mg/Liter, about 1.7 mg/Liter to about 1.9 mg/Liter, about 1.7 mg/Liter to about 2 mg/Liter, or about 1.9 mg/Liter to about 2 mg/Liter. In some embodiments, each vitamin present in the medium is present at about 0.1 mg/Liter, about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, about 1.9 mg/Liter, or about 2 mg/Liter. In some embodiments, each vitamin present in the medium is present at least about 0.1 mg/Liter, about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, or about 1.9 mg/Liter. In some embodiments, each vitamin present in the medium is present at most about 0.3 mg/Liter, about 0.5 mg/Liter, about 0.7 mg/Liter, about 0.9 mg/Liter, about 1.1 mg/Liter, about 1.3 mg/Liter, about 1.5 mg/Liter, about 1.7 mg/Liter, about 1.9 mg/Liter, or about 2 mg/Liter. In some embodiments, each vitamin present in the medium is present at about 10 mg/Liter to about 120 mg/Liter. In some embodiments, each vitamin present in the medium is present at about 10 mg/Liter to about 20 mg/Liter, about 10 mg/Liter to about 30 mg/Liter, about 10 mg/Liter to about 40 mg/Liter, about 10 mg/Liter to about 50 mg/Liter, about 10 mg/Liter to about 60 mg/Liter, about 10 mg/Liter to about 70 mg/Liter, about 10 mg/Liter to about 80 mg/Liter, about 10 mg/Liter to about 90 mg/Liter, about 10 mg/Liter to about 100 mg/Liter, about 10 mg/Liter to about 110 mg/Liter, about 10 mg/Liter to about 120 mg/Liter, about 20 mg/Liter to about 30 mg/Liter, about 20 mg/Liter to about 40 mg/Liter, about 20 mg/Liter to about 50 mg/Liter, about 20 mg/Liter to about 60 mg/Liter, about 20 mg/Liter to about 70 mg/Liter, about 20 mg/Liter to about 80 mg/Liter, about 20 mg/Liter to about 90 mg/Liter, about 20 mg/Liter to about 100 mg/Liter, about 20 mg/Liter to about 110 mg/Liter, about 20 mg/Liter to about 120 mg/Liter, about 30 mg/Liter to about 40 mg/Liter, about 30 mg/Liter to about 50 mg/Liter, about 30 mg/Liter to about 60 mg/Liter, about 30 mg/Liter to about 70 mg/Liter, about 30 mg/Liter to about 80 mg/Liter, about 30 mg/Liter to about 90 mg/Liter, about 30 mg/Liter to about 100 mg/Liter, about 30 mg/Liter to about 110 mg/Liter, about 30 mg/Liter to about 120 mg/Liter, about 40 mg/Liter to about 50 mg/Liter, about 40 mg/Liter to about 60 mg/Liter, about 40 mg/Liter to about 70 mg/Liter, about 40 mg/Liter to about 80 mg/Liter, about 40 mg/Liter to about 90 mg/Liter, about 40 mg/Liter to about 100 mg/Liter, about 40 mg/Liter to about 110 mg/Liter, about 40 mg/Liter to about 120 mg/Liter, about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 50 mg/Liter to about 110 mg/Liter, about 50 mg/Liter to about 120 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 60 mg/Liter to about 110 mg/Liter, about 60 mg/Liter to about 120 mg/Liter, about 70 mg/Liter to about 80 mg/Liter, about 70 mg/Liter to about 90 mg/Liter, about 70 mg/Liter to about 100 mg/Liter, about 70 mg/Liter to about 110 mg/Liter, about 70 mg/Liter to about 120 mg/Liter, about 80 mg/Liter to about 90 mg/Liter, about 80 mg/Liter to about 100 mg/Liter, about 80 mg/Liter to about 110 mg/Liter, about 80 mg/Liter to about 120 mg/Liter, about 90 mg/Liter to about 100 mg/Liter, about 90 mg/Liter to about 110 mg/Liter, about 90 mg/Liter to about 120 mg/Liter, about 100 mg/Liter to about 110 mg/Liter, about 100 mg/Liter to about 120 mg/Liter, or about 110 mg/Liter to about 120 mg/Liter. In some embodiments, each vitamin present in the medium is present at about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, about 110 mg/Liter, or about 120 mg/Liter. In some embodiments, each vitamin present in the medium is present at least about 10 mg/Liter, about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, or about 110 mg/Liter. In some embodiments, each vitamin present in the medium is present at most about 20 mg/Liter, about 30 mg/Liter, about 40 mg/Liter, about 50 mg/Liter, about 60 mg/Liter, about 70 mg/Liter, about 80 mg/Liter, about 90 mg/Liter, about 100 mg/Liter, about 110 mg/Liter, or about 120 mg/Liter.

In some embodiments, the glucose comprised in the medium is anhydrous. In some embodiments, the glucose is present in the L isoform. In some embodiments, the glucose is present in the D isoform. In some embodiments, the glucose is present in both isoforms. In some embodiments, glucose present in the medium is present at about 500 mg/Liter to about 1,600 mg/Liter. In some embodiments, glucose present in the medium is present at about 500 mg/Liter to about 600 mg/Liter, about 500 mg/Liter to about 700 mg/Liter, about 500 mg/Liter to about 800 mg/Liter, about 500 mg/Liter to about 900 mg/Liter, about 500 mg/Liter to about 1,000 mg/Liter, about 500 mg/Liter to about 1,100 mg/Liter, about 500 mg/Liter to about 1,200 mg/Liter, about 500 mg/Liter to about 1,300 mg/Liter, about 500 mg/Liter to about 1,400 mg/Liter, about 500 mg/Liter to about 1,500 mg/Liter, about 500 mg/Liter to about 1,600 mg/Liter, about 600 mg/Liter to about 700 mg/Liter, about 600 mg/Liter to about 800 mg/Liter, about 600 mg/Liter to about 900 mg/Liter, about 600 mg/Liter to about 1,000 mg/Liter, about 600 mg/Liter to about 1,100 mg/Liter, about 600 mg/Liter to about 1,200 mg/Liter, about 600 mg/Liter to about 1,300 mg/Liter, about 600 mg/Liter to about 1,400 mg/Liter, about 600 mg/Liter to about 1,500 mg/Liter, about 600 mg/Liter to about 1,600 mg/Liter, about 700 mg/Liter to about 800 mg/Liter, about 700 mg/Liter to about 900 mg/Liter, about 700 mg/Liter to about 1,000 mg/Liter, about 700 mg/Liter to about 1,100 mg/Liter, about 700 mg/Liter to about 1,200 mg/Liter, about 700 mg/Liter to about 1,300 mg/Liter, about 700 mg/Liter to about 1,400 mg/Liter, about 700 mg/Liter to about 1,500 mg/Liter, about 700 mg/Liter to about 1,600 mg/Liter, about 800 mg/Liter to about 900 mg/Liter, about 800 mg/Liter to about 1,000 mg/Liter, about 800 mg/Liter to about 1,100 mg/Liter, about 800 mg/Liter to about 1,200 mg/Liter, about 800 mg/Liter to about 1,300 mg/Liter, about 800 mg/Liter to about 1,400 mg/Liter, about 800 mg/Liter to about 1,500 mg/Liter, about 800 mg/Liter to about 1,600 mg/Liter, about 900 mg/Liter to about 1,000 mg/Liter, about 900 mg/Liter to about 1,100 mg/Liter, about 900 mg/Liter to about 1,200 mg/Liter, about 900 mg/Liter to about 1,300 mg/Liter, about 900 mg/Liter to about 1,400 mg/Liter, about 900 mg/Liter to about 1,500 mg/Liter, about 900 mg/Liter to about 1,600 mg/Liter, about 1,000 mg/Liter to about 1,100 mg/Liter, about 1,000 mg/Liter to about 1,200 mg/Liter, about 1,000 mg/Liter to about 1,300 mg/Liter, about 1,000 mg/Liter to about 1,400 mg/Liter, about 1,000 mg/Liter to about 1,500 mg/Liter, about 1,000 mg/Liter to about 1,600 mg/Liter, about 1,100 mg/Liter to about 1,200 mg/Liter, about 1,100 mg/Liter to about 1,300 mg/Liter, about 1,100 mg/Liter to about 1,400 mg/Liter, about 1,100 mg/Liter to about 1,500 mg/Liter, about 1,100 mg/Liter to about 1,600 mg/Liter, about 1,200 mg/Liter to about 1,300 mg/Liter, about 1,200 mg/Liter to about 1,400 mg/Liter, about 1,200 mg/Liter to about 1,500 mg/Liter, about 1,200 mg/Liter to about 1,600 mg/Liter, about 1,300 mg/Liter to about 1,400 mg/Liter, about 1,300 mg/Liter to about 1,500 mg/Liter, about 1,300 mg/Liter to about 1,600 mg/Liter, about 1,400 mg/Liter to about 1,500 mg/Liter, about 1,400 mg/Liter to about 1,600 mg/Liter, or about 1,500 mg/Liter to about 1,600 mg/Liter. In some embodiments, glucose present in the medium is present at about 500 mg/Liter, about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, about 1,500 mg/Liter, or about 1,600 mg/Liter. In some embodiments, glucose present in the medium is present at least about 500 mg/Liter, about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, or about 1,500 mg/Liter. In some embodiments, glucose present in the medium is present at most about 600 mg/Liter, about 700 mg/Liter, about 800 mg/Liter, about 900 mg/Liter, about 1,000 mg/Liter, about 1,100 mg/Liter, about 1,200 mg/Liter, about 1,300 mg/Liter, about 1,400 mg/Liter, about 1,500 mg/Liter, or about 1,600 mg/Liter.

In some embodiments, lipoic acid present in the medium is present at about 0.05 mg/Liter to about 0.5 mg/Liter. In some embodiments, the lipoic acid is present in the medium in the form of DL-thiotic acid. In some embodiments, lipoic acid present in the medium is present at about 0.05 mg/Liter to about 0.1 mg/Liter, about 0.05 mg/Liter to about 0.15 mg/Liter, about 0.05 mg/Liter to about 0.2 mg/Liter, about 0.05 mg/Liter to about 0.25 mg/Liter, about 0.05 mg/Liter to about 0.3 mg/Liter, about 0.05 mg/Liter to about 0.35 mg/Liter, about 0.05 mg/Liter to about 0.4 mg/Liter, about 0.05 mg/Liter to about 0.45 mg/Liter, about 0.05 mg/Liter to about 0.5 mg/Liter, about 0.1 mg/Liter to about 0.15 mg/Liter, about 0.1 mg/Liter to about 0.2 mg/Liter, about 0.1 mg/Liter to about 0.25 mg/Liter, about 0.1 mg/Liter to about 0.3 mg/Liter, about 0.1 mg/Liter to about 0.35 mg/Liter, about 0.1 mg/Liter to about 0.4 mg/Liter, about 0.1 mg/Liter to about 0.45 mg/Liter, about 0.1 mg/Liter to about 0.5 mg/Liter, about 0.15 mg/Liter to about 0.2 mg/Liter, about 0.15 mg/Liter to about 0.25 mg/Liter, about 0.15 mg/Liter to about 0.3 mg/Liter, about 0.15 mg/Liter to about 0.35 mg/Liter, about 0.15 mg/Liter to about 0.4 mg/Liter, about 0.15 mg/Liter to about 0.45 mg/Liter, about 0.15 mg/Liter to about 0.5 mg/Liter, about 0.2 mg/Liter to about 0.25 mg/Liter, about 0.2 mg/Liter to about 0.3 mg/Liter, about 0.2 mg/Liter to about 0.35 mg/Liter, about 0.2 mg/Liter to about 0.4 mg/Liter, about 0.2 mg/Liter to about 0.45 mg/Liter, about 0.2 mg/Liter to about 0.5 mg/Liter, about 0.25 mg/Liter to about 0.3 mg/Liter, about 0.25 mg/Liter to about 0.35 mg/Liter, about 0.25 mg/Liter to about 0.4 mg/Liter, about 0.25 mg/Liter to about 0.45 mg/Liter, about 0.25 mg/Liter to about 0.5 mg/Liter, about 0.3 mg/Liter to about 0.35 mg/Liter, about 0.3 mg/Liter to about 0.4 mg/Liter, about 0.3 mg/Liter to about 0.45 mg/Liter, about 0.3 mg/Liter to about 0.5 mg/Liter, about 0.35 mg/Liter to about 0.4 mg/Liter, about 0.35 mg/Liter to about 0.45 mg/Liter, about 0.35 mg/Liter to about 0.5 mg/Liter, about 0.4 mg/Liter to about 0.45 mg/Liter, about 0.4 mg/Liter to about 0.5 mg/Liter, or about 0.45 mg/Liter to about 0.5 mg/Liter. In some embodiments, lipoic acid present in the medium is present at about 0.05 mg/Liter, about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, about 0.45 mg/Liter, or about 0.5 mg/Liter. In some embodiments, lipoic acid present in the medium is present at least about 0.05 mg/Liter, about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, or about 0.45 mg/Liter. In some embodiments, lipoic acid present in the medium is present at most about 0.1 mg/Liter, about 0.15 mg/Liter, about 0.2 mg/Liter, about 0.25 mg/Liter, about 0.3 mg/Liter, about 0.35 mg/Liter, about 0.4 mg/Liter, about 0.45 mg/Liter, or about 0.5 mg/Liter.

In some embodiments, sodium bicarbonate present in the medium is present at about 250 mg/Liter to about 2,000 mg/Liter. In some embodiments, sodium bicarbonate present in the medium is present at about 250 mg/Liter to about 500 mg/Liter, about 250 mg/Liter to about 750 mg/Liter, about 250 mg/Liter to about 1,000 mg/Liter, about 250 mg/Liter to about 1,250 mg/Liter, about 250 mg/Liter to about 1,500 mg/Liter, about 250 mg/Liter to about 1,750 mg/Liter, about 250 mg/Liter to about 2,000 mg/Liter, about 500 mg/Liter to about 750 mg/Liter, about 500 mg/Liter to about 1,000 mg/Liter, about 500 mg/Liter to about 1,250 mg/Liter, about 500 mg/Liter to about 1,500 mg/Liter, about 500 mg/Liter to about 1,750 mg/Liter, about 500 mg/Liter to about 2,000 mg/Liter, about 750 mg/Liter to about 1,000 mg/Liter, about 750 mg/Liter to about 1,250 mg/Liter, about 750 mg/Liter to about 1,500 mg/Liter, about 750 mg/Liter to about 1,750 mg/Liter, about 750 mg/Liter to about 2,000 mg/Liter, about 1,000 mg/Liter to about 1,250 mg/Liter, about 1,000 mg/Liter to about 1,500 mg/Liter, about 1,000 mg/Liter to about 1,750 mg/Liter, about 1,000 mg/Liter to about 2,000 mg/Liter, about 1,250 mg/Liter to about 1,500 mg/Liter, about 1,250 mg/Liter to about 1,750 mg/Liter, about 1,250 mg/Liter to about 2,000 mg/Liter, about 1,500 mg/Liter to about 1,750 mg/Liter, about 1,500 mg/Liter to about 2,000 mg/Liter, or about 1,750 mg/Liter to about 2,000 mg/Liter. In some embodiments, sodium bicarbonate present in the medium is present at about 250 mg/Liter, about 500 mg/Liter, about 750 mg/Liter, about 1,000 mg/Liter, about 1,250 mg/Liter, about 1,500 mg/Liter, about 1,750 mg/Liter, or about 2,000 mg/Liter. In some embodiments, sodium bicarbonate present in the medium is present at least about 250 mg/Liter, about 500 mg/Liter, about 750 mg/Liter, about 1,000 mg/Liter, about 1,250 mg/Liter, about 1,500 mg/Liter, or about 1,750 mg/Liter. In some embodiments, sodium bicarbonate present in the medium is present at most about 500 mg/Liter, about 750 mg/Liter, about 1,000 mg/Liter, about 1,250 mg/Liter, about 1,500 mg/Liter, about 1,750 mg/Liter, or about 2,000 mg/Liter.

In some embodiments, sodium pyruvate present in the medium is present at about 50 mg/Liter to about 160 mg/Liter. In some embodiments, sodium pyruvate present in the medium is present at about 50 mg/Liter to about 60 mg/Liter, about 50 mg/Liter to about 70 mg/Liter, about 50 mg/Liter to about 80 mg/Liter, about 50 mg/Liter to about 90 mg/Liter, about 50 mg/Liter to about 100 mg/Liter, about 50 mg/Liter to about 110 mg/Liter, about 50 mg/Liter to about 120 mg/Liter, about 50 mg/Liter to about 130 mg/Liter, about 50 mg/Liter to about 140 mg/Liter, about 50 mg/Liter to about 150 mg/Liter, about 50 mg/Liter to about 160 mg/Liter, about 60 mg/Liter to about 70 mg/Liter, about 60 mg/Liter to about 80 mg/Liter, about 60 mg/Liter to about 90 mg/Liter, about 60 mg/Liter to about 100 mg/Liter, about 60 mg/Liter to about 110 mg/Liter, about 60 mg/Liter to about 120 mg/Liter, about 60 mg/Liter to about 130 mg/Liter, about 60 mg/Liter to about 140 mg/Liter, about 60 mg/Liter to about 150 mg/Liter, about 60 mg/Liter to about 160 mg/Liter, about 70 mg/Liter to about 80 mg/Liter, about 70 mg/Liter to about 90 mg/Liter, about 70 mg/Liter to about 100 mg/Liter, about 70 mg/Liter to about 110 mg/Liter, about 70 mg/Liter to about 120 mg/Liter, about 70 mg/Liter to about 130 mg/Liter, about 70 mg/Liter to about 140 mg/Liter, about 70 mg/Liter to about 150 mg/Liter, about 70 mg/Liter to about 160 mg/Liter, about 80 mg/Liter to about 90 mg/Liter, about 80 mg/Liter to about 100 mg/Liter, about 80 mg/Liter to about 110 mg/Liter, about 80 mg/Liter to about 120 mg/Liter, about 80 mg/Liter to about 130 mg/Liter, about 80 mg/Liter to about 140 mg/Liter, about 80 mg/Liter to about 150 mg/Liter, about 80 mg/Liter to about 160 mg/Liter, about 90 mg/Liter to about 100 mg/Liter, about 90 mg/Liter to about 110 mg/Liter, about 90 mg/Liter to about 120 mg/Liter, about 90 mg/Liter to about 130 mg/Liter, about 90 mg/Liter to about 140 mg/Liter, about 90 mg/Liter to about 150 mg/Liter, about 90 mg/Liter to about 160 mg/Liter, about 100 mg/Liter to about 110 mg/Liter, about 100 mg/Liter to about 120 mg/Liter, about 100 mg/Liter to about 130 mg/Liter, about 100 mg/Liter to about 140 mg/Liter, about 100 mg/Liter to about 150 mg/Liter, about 100 mg/Liter to about 160 mg/Liter, about 110 mg/Liter to about 120 mg/Liter, about 110 mg/Liter to about 130 mg/Liter, about 110 mg/Liter to about 140 mg/Liter, about 110 mg/Liter to about 150 mg/Liter, about 110 mg/Liter to about 160 mg/Liter, about 120 mg/Liter to about 130 mg/Liter, about 120 mg/Liter to about 140 mg/Liter, about 120 mg/Liter to about 150 mg/Liter, about 120 mg/Liter to about 160 mg/Liter, about 130 mg/Liter to about 140 mg/Liter, about 130 mg/Liter to about 150 mg/Liter, about 130 mg/Liter to about 160 mg/Liter, about 140 mg/Liter to about 150 mg/Liter, about 140 mg/Liter to about 160 mg/Liter, or about 150 mg/Liter to about 160 mg/Liter. In some embodiments, sodium pyruvate present in the medium is present at about 50 mg/Liter, about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, about 150 mg/Liter, or about 160 mg/Liter. In some embodiments, sodium pyruvate present in the medium is present at least about 50 mg/Liter, about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, or about 150 mg/Liter. In some embodiments, sodium pyruvate present in the medium is present at most about 60 mg/Liter, about 70 mg/Liter, about 80 mg/Liter, about 90 mg/Liter, about 100 mg/Liter, about 110 mg/Liter, about 120 mg/Liter, about 130 mg/Liter, about 140 mg/Liter, about 150 mg/Liter, or about 160 mg/Liter.

In some embodiments, the pH of the alpha MEM is between 7.0 and 7.4.

In some embodiments, the alpha MEM comprises the ingredients are presented in

TABLE 1
Table 1

| Components | mg/Liter | Components | mg/Liter |
|---|---|---|---|
| INORGANIC SALTS | | AMINO ACIDS (CONTINUED) | |
| Calcium Chloride, dihydrate (CaCl₂.2H₂0) | 264.00 | L-Threonine (C₄H₉NO₃) | 48.0000 |
| Magnesium Sulfate, heptahydrate | 200.00 | L-Tryptophan (C11H12N2⁰2) | 10.0000 |
| Potassium Chloride (KCI) | 400.00 | L-Tyrosine (C₉H₉NO₃) | 36.0000 |
| Sodium Chloride (NaCI) | 6800.0 | L-Valine (C₅H_{1 1}N0₇) | 46.0000 |
| Sodium Phosphate Monobasic, dihydrate | 158.00 | | |
| [symbol] | | | |
| OTHER COMPONENTS | | VITAMINS | |
| D-Glucose, Anhydrous (C₆11₁₂0₆) | 1000.00 | L-Ascorbic Acid (C6H806) | 50.0000 |
| Lipoic Acid (DL-Thiotic Acid) [symbol] | 0.20000 | D-Biotin (CᵢₒHᵢ₆N₂0₃S) | 0.1000 |
| Sodium Bicarbonate (NaHCO₃) | 2200.00 | Choline Chloride (C₅H₁₄CIN0) | 1.0000 |
| Sodium Pyruvate (C₃H₃Na0₃) | 110.000 | D-Calcium Pantothenate | 1.0000 |
| | | Folic Acid (C191⁻119N706) | 1.0000 |
| AMINO ACIDS | | Myo-Inositol (C₆H₁₂0₆) | 2.0000 |
| Glycine (C2H5NO2) | 50.0000 | Niacinamide (C₆H₆N₂0) | 1.0000 |
| L-Alanine (C3H7N021 | 25.0000 | Pyridoxal HCI (C₈H₉NO₃ · FICI) | 1.0000 |
| L-Alanyl-Glutamine (C₈H₁₅N₃0₄) | 406.000 | Pyruvic Acid, Sodium Salt | 110.00 |
| L-Arginine HCI (C₆H₁₅CIN₄0₂) | 105.000 | Riboflavin (C₁₇H_{2O}N₄0₆) | 0.1000 |
| L-Asparagine. monohvdrate (C₄H₈N₂0₃ [symbol] | 50.0000 | Thimaine HCI (C₁₂H₁₇CIN₄0S · HCI) | 1.0000 |
| L-Aspartic Acid (C4H7N04) | 30.0000 | Vitamin B12 (C63H88CON14°14P) | 1.3600 |
| L-Cvsteine HCI. monohydrate | 100.000 | | |
| L-Cystine (C6Hi2N20452) | 24.0000 | | |
| L-Glutamic Acid (C₅H₉N0₄) | 75.0000 | | |
| L-Histidine (C6H9N302) | 31.0000 | | |
| L-Isoleucine (C₆F1₁₃NO₂) | 52.4000 | | |
| L-Leucine (C₆F1₁₃NO₂) | 52.4000 | | |
| L-Lysine (C₆H₁₄N₂0₂) | 58.0000 | | |
| L-Methionine (C₅H₁₁NO₂S) | 15.0000 | | |
| L-Phenylalanine (C₉H₁₁NO₂) | 32.0000 | | |
| L-Proline (C₅H₉NO2) | 40.0000 | | |
| L-Serine (C₃H₇NO₃) | 25.0000 | | |

[symbol]

indicates data missing or illegible when filed

In some embodiments, the vBA-MSCs are cultured in a medium comprising alpha MEM as described in Table 1, 10% Stemulate hPL (no heparin required), 2 ng/mL recombinant, carrier free FGF, and 2 ng/mL recombinant, carrier free EGF.

In some embodiments, the primary MSCs may be further passaged to non-primary cells (e.g. removed from the culture surface and expanded into additional area) by seeding at a density of about 1,000 to about one million nucleated cells/cm² of culture dish (e.g. about 5,900 cells/cm² plus and minus about 1,200), and then culturing for additional days, e.g. about 14±about 2 days. In suitable embodiments, the primary cells may be grown to confluence, and in some instances may be passaged to a second culture of non-primary cells by seeding the primary cells from a confluent primary cell culture in the second culture surface in an amount below confluence and growing the non-primary culture to confluence. This method can be repeated for additional passages.

In some embodiments, the MSCs in the treatment composition may originate from sequential generation number (i.e., they are within about 1 or about 2 or about 3 or about 4 cell doublings of each other). Optionally, the average number of cell doublings in the present composition treatment composition may be about 20 to about 25 doublings. Optionally, the average number of cell doublings in the present treatment composition may be about 9 to about 13 (e.g., about 11 or about 11.2) doublings arising from the primary culture, plus about 1, about 2, about 3, or about 4 doublings per passage (for example, about 2.5 doublings per passage). Exemplary average cell doublings in present preparations may be of about 13.5, about 16, about 18.5, about 21, about 23.5, about 26, about 28.5, about 31, about 33.5, or about 36 when produced by about 1, about 2, about 3, about 4, about 5, about 6, about 7, about 8, about 9, or about 10 passages, respectively.

In some embodiments, notwithstanding one or more population doublings, the MSCs in the treatment composition (e.g. vBA-MSCs) may originate from MSCs that were cultured through about 1, about 2, about 3, about 4, about 5, about 6, about 7, about 8, about 9, or about 10 passages.

In some embodiments, the preparations and compositions of the present disclosure may comprise at least 100 million vBA-MSCs having an antigen profile of more than about 1.75% CD45+ cells, at least about 95% CD105+ cells, and at least about 95% CD166+ cells and the cells may be expanded ex vivo from passage 2 until passage 4 while maintaining population uniformity based upon the antigen profile (i.e. more than about 1.75% CD45+ cells, at least about 95% CD105+ cells, and at least about 95% CD166+ cells).

In some embodiments, the preparations and compositions of the present disclosure may comprise vBA-MSCs having an antigen profile of reduced expression of one or more senescent cell markers, as compared to bone marrow-derived MSCs prepared according to known MSC culturing techniques. In some embodiments, the one or more senescent cell markers comprise MIC-A, MIC-B, ULBP2, or any combination thereof. NK cell-mediated immune responses are stimulated by MIC-A, MIC-B, and/or ULBP2.

In some embodiments, the vBA-MSC preparations and compositions described herein comprise an amount of cells that express one or more senescent cell markers of about 1% less than bone marrow-derived MSCs to about 100% less than bone marrow-derived MSCs. In some embodiments, the vBA-MSC preparations and compositions described herein comprise an amount of cells that express one or more senescent cell markers of about 100% less than bone marrow-derived MSCs to about 90% less than bone marrow-derived MSCs, about 100% less than bone marrow-derived MSCs to about 80% less than bone marrow-derived MSCs, about 100% less than bone marrow-derived MSCs to about 70% less than bone marrow-derived MSCs, about 100% less than bone marrow-derived MSCs to about 60% less than bone marrow-derived MSCs, about 100% less than bone marrow-derived MSCs to about 50% less than bone marrow-derived MSCs, about 100% less than bone marrow-derived MSCs to about 40% less than bone marrow-derived MSCs, about 100% less than bone marrow-derived MSCs to about 30% less than bone marrow-derived MSCs, about 100% less than bone marrow-derived MSCs to about 20% less than bone marrow-derived MSCs, about 100% less than bone marrow-derived MSCs to about 10% less than bone marrow-derived MSCs, about 100% less than bone marrow-derived MSCs to about 5% less than bone marrow-derived MSCs, about 100% less than bone marrow-derived MSCs to about 1% less than bone marrow-derived MSCs, about 90% less than bone marrow-derived MSCs to about 80% less than bone marrow-derived MSCs, about 90% less than bone marrow-derived MSCs to about 70% less than bone marrow-derived MSCs, about 90% less than bone marrow-derived MSCs to about 60% less than bone marrow-derived MSCs, about 90% less than bone marrow-derived MSCs to about 50% less than bone marrow-derived MSCs, about 90% less than bone marrow-derived MSCs to about 40% less than bone marrow-derived MSCs, about 90% less than bone marrow-derived MSCs to about 30% less than bone marrow-derived MSCs, about 90% less than bone marrow-derived MSCs to about 20% less than bone marrow-derived MSCs, about 90% less than bone marrow-derived MSCs to about 10% less than bone marrow-derived MSCs, about 90% less than bone marrow-derived MSCs to about 5% less than bone marrow-derived MSCs, about 90% less than bone marrow-derived MSCs to about 1% less than bone marrow-derived MSCs, about 80% less than bone marrow-derived MSCs to about 70% less than bone marrow-derived MSCs, about 80% less than bone marrow-derived MSCs to about 60% less than bone marrow-derived MSCs, about 80% less than bone marrow-derived MSCs to about 50% less than bone marrow-derived MSCs, about 80% less than bone marrow-derived MSCs to about 40% less than bone marrow-derived MSCs, about 80% less than bone marrow-derived MSCs to about 30% less than bone marrow-derived MSCs, about 80% less than bone marrow-derived MSCs to about 20% less than bone marrow-derived MSCs, about 80% less than bone marrow-derived MSCs to about 10% less than bone marrow-derived MSCs, about 80% less than bone marrow-derived MSCs to about 5% less than bone marrow-derived MSCs, about 80% less than bone marrow-derived MSCs to about 1% less than bone marrow-derived MSCs, about 70% less than bone marrow-derived MSCs to about 60% less than bone marrow-derived MSCs, about 70% less than bone marrow-derived MSCs to about 50% less than bone marrow-derived MSCs, about 70% less than bone marrow-derived MSCs to about 40% less than bone marrow-derived MSCs, about 70% less than bone marrow-derived MSCs to about 30% less than bone marrow-derived MSCs, about 70% less than bone marrow-derived MSCs to about 20% less than bone marrow-derived MSCs, about 70% less than bone marrow-derived MSCs to about 10% less than bone marrow-derived MSCs, about 70% less than bone marrow-derived MSCs to about 5% less than bone marrow-derived MSCs, about 70% less than bone marrow-derived MSCs to about 1% less than bone marrow-derived MSCs, about 60% less than bone marrow-derived MSCs to about 50% less than bone marrow-derived MSCs, about 60% less than bone marrow-derived MSCs to about 40% less than bone marrow-derived MSCs, about 60% less than bone marrow-derived MSCs to about 30% less than bone marrow-derived MSCs, about 60% less than bone marrow-derived MSCs to about 20% less than bone marrow-derived MSCs, about 60% less than bone marrow-derived MSCs to about 10% less than bone marrow-derived MSCs, about 60% less than bone marrow-derived MSCs to about 5% less than bone marrow-derived MSCs, about 60% less than bone marrow-derived MSCs to about 1% less than bone marrow-derived MSCs, about 50% less than bone marrow-derived MSCs to about 40% less than bone marrow-derived MSCs, about 50% less than bone marrow-derived MSCs to about 30% less than bone marrow-derived MSCs, about 50% less than bone marrow-derived MSCs to about 20% less than bone marrow-derived MSCs, about 50% less than bone marrow-derived MSCs to about 10% less than bone marrow-derived MSCs, about 50% less than bone marrow-derived MSCs to about 5% less than bone marrow-derived MSCs, about 50% less than bone marrow-derived MSCs to about 1% less than bone marrow-derived MSCs, about 40% less than bone marrow-derived MSCs to about 30% less than bone marrow-derived MSCs, about 40% less than bone marrow-derived MSCs to about 20% less than bone marrow-derived MSCs, about 40% less than bone marrow-derived MSCs to about 10% less than bone marrow-derived MSCs, about 40% less than bone marrow-derived MSCs to about 5% less than bone marrow-derived MSCs, about 40% less than bone marrow-derived MSCs to about 1% less than bone marrow-derived MSCs, about 30% less than bone marrow-derived MSCs to about 20% less than bone marrow-derived MSCs, about 30% less than bone marrow-derived MSCs to about 10% less than bone marrow-derived MSCs, about 30% less than bone marrow-derived MSCs to about 5% less than bone marrow-derived MSCs, about 30% less than bone marrow-derived MSCs to about 1% less than bone marrow-derived MSCs, about 20% less than bone marrow-derived MSCs to about 10% less than bone marrow-derived MSCs, about 20% less than bone marrow-derived MSCs to about 5% less than bone marrow-derived MSCs, about 20% less than bone marrow-derived MSCs to about 1% less than bone marrow-derived MSCs, about 10% less than bone marrow-derived MSCs to about 5% less than bone marrow-derived MSCs, about 10% less than bone marrow-derived MSCs to about 1% less than bone marrow-derived MSCs, or about 5% less than bone marrow-derived MSCs to about 1% less than bone marrow-derived MSCs. In some embodiments, the vBA-MSC preparations and compositions described herein comprise an amount of cells that express one or more senescent cell markers of about 100% less than bone marrow-derived MSCs, about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, about 5% less than bone marrow-derived MSCs, or about 1% less than bone marrow-derived MSCs. In some embodiments, the vBA-MSC preparations and compositions described herein comprise an amount of cells that express one or more senescent cell markers of at least about 100% less than bone marrow-derived MSCs, about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, or about 5% less than bone marrow-derived MSCs. In some embodiments, the vBA-MSC preparations and compositions described herein comprise an amount of cells that express one or more senescent cell markers of at most about 90% less than bone marrow-derived MSCs, about 80% less than bone marrow-derived MSCs, about 70% less than bone marrow-derived MSCs, about 60% less than bone marrow-derived MSCs, about 50% less than bone marrow-derived MSCs, about 40% less than bone marrow-derived MSCs, about 30% less than bone marrow-derived MSCs, about 20% less than bone marrow-derived MSCs, about 10% less than bone marrow-derived MSCs, about 5% less than bone marrow-derived MSCs, or about 1% less than bone marrow-derived MSCs.

### Thawing and Storage

Provided herein, in some embodiments, is a method of warming a cryopreserved population of stem cells to a first temperature and storing said stem cells at a second temperature less than about 40° C. In some embodiments, the first temperature that the cryopreserved population of stem cells is warmed to is greater than about 0° C. In some embodiments, the first temperature is greater than about 20° C. In some embodiments, the second temperature that the stem cells are stored at is a hypothermic temperature.

Provided herein, in one aspect, is a method for preparing stem cells for infusion, the method comprising: (a) providing a cryopreserved population of cells comprising said stem cells; (b) warming said stem cells to a first temperature and holding said stem cells at said first temperature for a first period of time; and (c) changing said first temperature to a second temperature and maintaining said stem cells at said second temperature for a second period of time.

In some embodiments, said first temperature is greater than about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said first temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said first temperature is greater than 0° C.

In some embodiments, said second temperature is less than about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said second temperature is about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C. In some embodiments, said second temperature is hypothermic. In some embodiments, said second temperature is less than 40° C.

In some embodiments, said first time period is less than about one week. In some embodiments, said time period is less than about 7 days, about 6 days, about 5 days, about 4 days, about 3 days, about 2 days, or about 1 day. In some embodiments, said time period is less than about 5 days. In some embodiments, said time period is less than about 2 days. In some embodiments, said time period is less than about 1 day. In some embodiments, said time period is less than about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or about 1 hour. In some embodiments, said time period is less than about 12 hours. In some embodiments, said time period is less than about 6 hours. In some embodiments, said time period is less than about 2 hours. In some embodiments, said time period is less than about 60 minutes, about 59 minutes, about 58 minutes, about 57 minutes, about 56 minutes, about 55 minutes, about 54 minutes, about 53 minutes, about 52 minutes, about 51 minutes, about 50 minutes, about 49 minutes, about 48 minutes, about 47 minutes, about 46 minutes, about 45 minutes, about 44 minutes, about 43 minutes, about 42 minutes, about 41 minutes, about 40 minutes, about 39 minutes, about 38 minutes, about 37 minutes, about 36 minutes, about 35 minutes, about 34 minutes, about 33 minutes, about 32 minutes, about 31 minutes, about 30 minutes, about 29 minutes, about 28 minutes, about 27 minutes, about 26 minutes, about 25 minutes, about 24 minutes, about 23 minutes, about 22 minutes, about 21 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, or about 1 minute.

In some embodiments, the stem cells are cultured after they are warmed/thawed. In some embodiments, the culturing is not accounted for in the first period of time described herein. In some embodiments, the culturing is accounted for in the first period of time. In some embodiments, the culturing follows the MSC culturing methods described herein. In some embodiments, the post-thaw culturing methods comprise not allowing the MSCs to double in population. In some embodiments, the MSCs are recovered and/or packaged prior to doubling. In some embodiments, the post-thaw culturing methods comprise culturing said stem cells at about 10,000 to about 50,000, about 10,000 to about 40,000, about 10,000 to about 30,000, about 10,000 to about 20,000, about 20,000 to about 50,000, about 20,000 to about 40,000, about 20,000 to about 30,000, about 30,000 to about 50,000, about 30,000 to about 40,000, or about 40,000 to about 50,000 cells/cm² at said first temperature. In some embodiments, for a method provided herein, the method further comprises, prior to (c), culturing said stem cells at about 10,000 cells/cm² to about 50,000 cells/cm² at said first temperature. In some embodiments, for a method provided herein, the method further comprises, prior to (c), culturing said stem cells at about 10,000, about 20,000, about 30,000, about 40,000, or about 50,000 cells/cm² at said first temperature. In some embodiments, for a method provided herein, the method further comprises, prior to (c), culturing said stem cells at about 32,000 cells/cm² at said first temperature. In some embodiments, the post-thaw culturing takes place in a T25 flask. In some embodiments, the post-thaw culturing takes place in a T75 flask. In some embodiments, the post-thaw culturing takes place in a other sized flasks.

In some embodiments, the stem cells are packaged in 5 mL volumes of 10×10⁶ live cells/mL in Plasma-Lyte A+2.5% HSA (Rinse Media). In some embodiments, the stem cells are packaged in volumes of about 1×10⁶ live cells/mL to about 12×10⁶ live cells/mL. In some embodiments, the stem cells are packaged in volumes of about 1×10\hat{ }6 live cells/mL to about 2×10⁶ live cells/mL, about 1×10⁶ live cells/mL to about 3×10⁶ live cells/mL, about 1×10⁶ live cells/mL to about 4×10⁶ live cells/mL, about 1×10⁶ live cells/mL to about 5×10⁶ live cells/mL, about 1×10⁶ live cells/mL to about 6×10⁶ live cells/mL, about 1×10⁶ live cells/mL to about 7×10⁶ live cells/mL, about 1×10⁶ live cells/mL to about 8×10⁶ live cells/mL, about 1×10⁶ live cells/mL to about 9×10⁶ live cells/mL, about 1×10⁶ live cells/mL to about 10×10⁶ live cells/mL, about 1×10⁶ live cells/mL to about 11×10⁶ live cells/mL, about 1×10⁶ live cells/mL to about 12×10⁶ live cells/mL, about 2×10⁶ live cells/mL to about 3×10⁶ live cells/mL, about 2×10⁶ live cells/mL to about 4×10⁶ live cells/mL, about 2×10⁶ live cells/mL to about 5×10⁶ live cells/mL, about 2×10⁶ live cells/mL to about 6×10⁶ live cells/mL, about 2×10⁶ live cells/mL to about 7×10⁶ live cells/mL, about 2×10⁶ live cells/mL to about 8×10⁶ live cells/mL, about 2×10⁶ live cells/mL to about 9×10⁶ live cells/mL, about 2×10⁶ live cells/mL to about 10×10⁶ live cells/mL, about 2×10⁶ live cells/mL to about 11×10⁶ live cells/mL, about 2×10⁶ live cells/mL to about 12×10⁶ live cells/mL, about 3×10⁶ live cells/mL to about 4×10⁶ live cells/mL, about 3×10⁶ live cells/mL to about 5×10⁶ live cells/mL, about 3×10⁶ live cells/mL to about 6×10⁶ live cells/mL, about 3×10⁶ live cells/mL to about 7×10⁶ live cells/mL, about 3×10⁶ live cells/mL to about 8×10⁶ live cells/mL, about 3×10⁶ live cells/mL to about 9×10⁶ live cells/mL, about 3×10⁶ live cells/mL to about 10×10⁶ live cells/mL, about 3×10⁶ live cells/mL to about 11×10⁶ live cells/mL, about 3×10⁶ live cells/mL to about 12×10⁶ live cells/mL, about 4×10⁶ live cells/mL to about 5×10⁶ live cells/mL, about 4×10⁶ live cells/mL to about 6×10⁶ live cells/mL, about 4×10⁶ live cells/mL to about 7×10⁶ live cells/mL, about 4×10⁶ live cells/mL to about 8×10⁶ live cells/mL, about 4×10⁶ live cells/mL to about 9×10⁶ live cells/mL, about 4×10⁶ live cells/mL to about 10×10⁶ live cells/mL, about 4×10⁶ live cells/mL to about 11×10⁶ live cells/mL, about 4×10⁶ live cells/mL to about 12×10\hat{ }6 live cells/mL, about 5×10\hat{ }6 live cells/mL to about 6×10\hat{ }6 live cells/mL, about 5×10⁶ live cells/mL to about 7×10⁶ live cells/mL, about 5×10⁶ live cells/mL to about 8×10⁶ live cells/mL, about 5×10⁶ live cells/mL to about 9×10⁶ live cells/mL, about 5×10⁶ live cells/mL to about 10×10⁶ live cells/mL, about 5×10⁶ live cells/mL to about 11×10⁶ live cells/mL, about 5×10⁶ live cells/mL to about 12×10⁶ live cells/mL, about 6×10⁶ live cells/mL to about 7×10⁶ live cells/mL, about 6×10⁶ live cells/mL to about 8×10⁶ live cells/mL, about 6×10⁶ live cells/mL to about 9×10⁶ live cells/mL, about 6×10⁶ live cells/mL to about 10×10⁶ live cells/mL, about 6×10⁶ live cells/mL to about 11×10⁶ live cells/mL, about 6×10⁶ live cells/mL to about 12×10⁶ live cells/mL, about 7×10⁶ live cells/mL to about 8×10⁶ live cells/mL, about 7×10⁶ live cells/mL to about 9×10⁶ live cells/mL, about 7×10⁶ live cells/mL to about 10×10⁶ live cells/mL, about 7×10⁶ live cells/mL to about 11×10⁶ live cells/mL, about 7×10⁶ live cells/mL to about 12×10⁶ live cells/mL, about 8×10⁶ live cells/mL to about 9×10⁶ live cells/mL, about 8×10⁶ live cells/mL to about 10×10⁶ live cells/mL, about 8×10⁶ live cells/mL to about 11×10⁶ live cells/mL, about 8×10⁶ live cells/mL to about 12×10⁶ live cells/mL, about 9×10⁶ live cells/mL to about 10×10⁶ live cells/mL, about 9×10⁶ live cells/mL to about 11×10⁶ live cells/mL, about 9×10⁶ live cells/mL to about 12×10⁶ live cells/mL, about 10×10⁶ live cells/mL to about 11×10⁶ live cells/mL, about 10×10⁶ live cells/mL to about 12×10⁶ live cells/mL, or about 11×10⁶ live cells/mL to about 12×10⁶ live cells/mL. In some embodiments, the stem cells are packaged in volumes of about 1×10⁶ live cells/mL, about 2×10⁶ live cells/mL, about 3×10⁶ live cells/mL, about 4×10⁶ live cells/mL, about 5×10⁶ live cells/mL, about 6×10⁶ live cells/mL, about 7×10⁶ live cells/mL, about 8×10⁶ live cells/mL, about 9×10⁶ live cells/mL, about 10×10⁶ live cells/mL, about 11×10⁶ live cells/mL, or about 12×10⁶ live cells/mL. In some embodiments, the stem cells are packaged in volumes of at least about 1×10⁶ live cells/mL, about 2×10⁶ live cells/mL, about 3×10⁶ live cells/mL, about 4×10⁶ live cells/mL, about 5×10⁶ live cells/mL, about 6×10⁶ live cells/mL, about 7×10⁶ live cells/mL, about 8×10⁶ live cells/mL, about 9×10⁶ live cells/mL, about 10×10⁶ live cells/mL, or about 11×10⁶ live cells/mL. In some embodiments, the stem cells are packaged in volumes of at most about 2×10⁶ live cells/mL, about 3×10⁶ live cells/mL, about 4×10⁶ live cells/mL, about 5×10⁶ live cells/mL, about 6×10⁶ live cells/mL, about 7×10⁶ live cells/mL, about 8×10⁶ live cells/mL, about 9×10⁶ live cells/mL, about 10×10⁶ live cells/mL, about 11×10⁶ live cells/mL, or about 12×10⁶ live cells/mL.

In some embodiments, the live stem cells are packaged in vials comprising about 1 mL to about 12 mL. In some embodiments, the live stem cells are packaged in vials comprising about 1 mL to about 2 mL, about 1 mL to about 3 mL, about 1 mL to about 4 mL, about 1 mL to about 5 mL, about 1 mL to about 6 mL, about 1 mL to about 7 mL, about 1 mL to about 8 mL, about 1 mL to about 9 mL, about 1 mL to about 10 mL, about 1 mL to about 11 mL, about 1 mL to about 12 mL, about 2 mL to about 3 mL, about 2 mL to about 4 mL, about 2 mL to about 5 mL, about 2 mL to about 6 mL, about 2 mL to about 7 mL, about 2 mL to about 8 mL, about 2 mL to about 9 mL, about 2 mL to about 10 mL, about 2 mL to about 11 mL, about 2 mL to about 12 mL, about 3 mL to about 4 mL, about 3 mL to about 5 mL, about 3 mL to about 6 mL, about 3 mL to about 7 mL, about 3 mL to about 8 mL, about 3 mL to about 9 mL, about 3 mL to about 10 mL, about 3 mL to about 11 mL, about 3 mL to about 12 mL, about 4 mL to about 5 mL, about 4 mL to about 6 mL, about 4 mL to about 7 mL, about 4 mL to about 8 mL, about 4 mL to about 9 mL, about 4 mL to about 10 mL, about 4 mL to about 11 mL, about 4 mL to about 12 mL, about 5 mL to about 6 mL, about 5 mL to about 7 mL, about 5 mL to about 8 mL, about 5 mL to about 9 mL, about 5 mL to about 10 mL, about 5 mL to about 11 mL, about 5 mL to about 12 mL, about 6 mL to about 7 mL, about 6 mL to about 8 mL, about 6 mL to about 9 mL, about 6 mL to about 10 mL, about 6 mL to about 11 mL, about 6 mL to about 12 mL, about 7 mL to about 8 mL, about 7 mL to about 9 mL, about 7 mL to about 10 mL, about 7 mL to about 11 mL, about 7 mL to about 12 mL, about 8 mL to about 9 mL, about 8 mL to about 10 mL, about 8 mL to about 11 mL, about 8 mL to about 12 mL, about 9 mL to about 10 mL, about 9 mL to about 11 mL, about 9 mL to about 12 mL, about 10 mL to about 11 mL, about 10 mL to about 12 mL, or about 11 mL to about 12 mL. In some embodiments, the live stem cells are packaged in vials comprising about 1 mL, about 2 mL, about 3 mL, about 4 mL, about 5 mL, about 6 mL, about 7 mL, about 8 mL, about 9 mL, about 10 mL, about 11 mL, or about 12 mL. In some embodiments, the live stem cells are packaged in vials comprising at least about 1 mL, about 2 mL, about 3 mL, about 4 mL, about 5 mL, about 6 mL, about 7 mL, about 8 mL, about 9 mL, about 10 mL, or about 11 mL. In some embodiments, the live stem cells are packaged in vials comprising at most about 2 mL, about 3 mL, about 4 mL, about 5 mL, about 6 mL, about 7 mL, about 8 mL, about 9 mL, about 10 mL, about 11 mL, or about 12 mL.

Once the stem cells are packaged, the stem cells are put under the second temperature for the second time period. In some embodiments, the second temperature is less than about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In some embodiments, said cell culture is maintained at about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In some embodiments, the second temperature is less than 37° C. In some embodiments, the second temperature is less than 35° C. In some embodiments, the second temperature is less than 30° C. In some embodiments, the second temperature is less than 25° C. In some embodiments, said the second temperature is less than 20° C. In some embodiments, the second temperature is about 2° C. to about 8° C.

In some embodiments, said second time period is less than about one week. In some embodiments, said time period is less than about 7 days, about 6 days, about 5 days, about 4 days, about 3 days, about 2 days, or about 1 day. In some embodiments, said time period is less than about 5 days. In some embodiments, said time period is less than about 2 days. In some embodiments, said time period is less than about 1 day. In some embodiments, said time period is less than about 24 hours, about 23 hours, about 22 hours, about 21 hours, about 20 hours, about 19 hours, about 18 hours, about 17 hours, about 16 hours, about 15 hours, about 14 hours, about 13 hours, about 12 hours, about 11 hours, about 10 hours, about 9 hours, about 8 hours, about 7 hours, about 6 hours, about 5 hours, about 4 hours, about 3 hours, about 2 hours, or about 1 hour. In some embodiments, said time period is less than about 12 hours. In some embodiments, said time period is less than about 6 hours. In some embodiments, said time period is less than about 2 hours. In some embodiments, said time period is less than about 60 minutes, about 59 minutes, about 58 minutes, about 57 minutes, about 56 minutes, about 55 minutes, about 54 minutes, about 53 minutes, about 52 minutes, about 51 minutes, about 50 minutes, about 49 minutes, about 48 minutes, about 47 minutes, about 46 minutes, about 45 minutes, about 44 minutes, about 43 minutes, about 42 minutes, about 41 minutes, about 40 minutes, about 39 minutes, about 38 minutes, about 37 minutes, about 36 minutes, about 35 minutes, about 34 minutes, about 33 minutes, about 32 minutes, about 31 minutes, about 30 minutes, about 29 minutes, about 28 minutes, about 27 minutes, about 26 minutes, about 25 minutes, about 24 minutes, about 23 minutes, about 22 minutes, about 21 minutes, about 20 minutes, about 19 minutes, about 18 minutes, about 17 minutes, about 16 minutes, about 15 minutes, about 14 minutes, about 13 minutes, about 12 minutes, about 11 minutes, about 10 minutes, about 9 minutes, about 8 minutes, about 7 minutes, about 6 minutes, about 5 minutes, about 4 minutes, about 3 minutes, about 2 minutes, or about 1 minute.

Provided herein, in another aspect, is a method for preparing a sample of stem cells for direct infusion, the method comprising:
a. providing a cell culture comprising said sample of stem cells wherein said cell culture was cryopreserved and passaged;
b. thawing said cell culture; and
c. maintaining said cell culture at less than 40° C.

In some embodiments, subsequent to (b) said cell culture is not doubled.

In some embodiments, said cell culture is maintained at hypothermic conditions. In some embodiments, said cell culture is maintained at less than about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In some embodiments, said cell culture is maintained at about 40° C., about 39° C., about 38° C., about 37° C., about 36° C., about 35° C., about 34° C., about 33° C., about 32° C., about 31° C., about 30° C., about 29° C., about 28° C., about 27° C., about 26° C., about 25° C., about 24° C., about 23° C., about 22° C., about 21° C., about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., or about 1° C. In some embodiments, said cell culture is maintained at less than 37° C. In some embodiments, said cell culture is maintained at less than 35° C. In some embodiments, said cell culture is maintained at less than 30° C. In some embodiments, said cell culture is maintained at less than 25° C. In some embodiments, said cell culture is maintained at less than 20° C. In some embodiments, said cell culture is maintained at about 2° C. to about 8° C.

In some embodiments, said thawing occurs at about 10° C. to about 100° C., about 10° C. to about 90° C., about 10° C. to about 80° C., about 10° C. to about 70° C., about 10° C. to about 60° C., about 10° C. to about 50° C., about 10° C. to about 40° C., about 10° C. to about 30° C., about 10° C. to about 20° C., about 20° C. to about 100° C., about 20° C. to about 90° C., about 20° C. to about 80° C., about 20° C. to about 70° C., about 20° C. to about 60° C., about 20° C. to about 50° C., about 20° C. to about 40° C., about 20° C. to about 30° C., about 30° C. to about 100° C., about 30° C. to about 90° C., about 30° C. to about 80° C., about 30° C. to about 70° C., about 30° C. to about 60° C., about 30° C. to about 50° C., about 30° C. to about 40° C., about 40° C. to about 100° C., about 40° C. to about 90° C., about 40° C. to about 80° C., about 40° C. to about 70° C., about 40° C. to about 60° C., about 40° C. to about 50° C., about 50° C. to about 100° C., about 50° C. to about 90° C., about 50° C. to about 80° C., about 50° C. to about 70° C., about 50° C. to about 60° C., about 60° C. to about 100° C., about 60° C. to about 90° C., about 60° C. to about 80° C., about 60° C. to about 70° C., about 70° C. to about 100° C., about 70° C. to about 90° C., about 70° C. to about 80° C., about 80° C. to about 100° C., about 80° C. to about 90° C., or about 90° C. to about 100° C. In some embodiments, said thawing occurs at about 30° C. to about 40° C. In some embodiments, said thawing occurs at about 30° C. In some embodiments, said thawing occurs at about 31° C. In some embodiments, said thawing occurs at about 32° C. In some embodiments, said thawing occurs at about 33° C. In some embodiments, said thawing occurs at about 34° C. In some embodiments, said thawing occurs at about 35° C. In some embodiments, said thawing occurs at about 36° C. In some embodiments, said thawing occurs at about 37° C. In some embodiments, said thawing occurs at about 38° C. In some embodiments, said thawing occurs at about 39° C. In some embodiments, said thawing occurs at about 40° C.

In some embodiments, said cell culture is maintained at less than room temperature for at least about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, or about 60 minutes. In some embodiments, said cell culture is maintained at less than room temperature for at least about 30 minutes. In some embodiments, said cell culture is maintained at less than room temperature for at least about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, or about 50 hours. In some embodiments, said cell culture is maintained at less than room temperature for at least 12 hours. In some embodiments, said cell culture is maintained at less than room temperature for at least 24 hours. In some embodiments, said cell culture is maintained at less than room temperature for at least 48 hours.

Provided herein, in another aspect, is a method for preparing a sample of stem cells for direct infusion, the method comprising:
a. providing a cell culture comprising said sample of stem cells wherein said cell culture was cryopreserved and passaged;
b. thawing said cell culture; and
c. maintaining said cell culture at greater than 0° C.

In some embodiments, said cell culture is maintained at greater than about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In some embodiments, said cell culture is maintained at about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C. In some embodiments, said cell culture is maintained at hypothermic conditions. In some embodiments, said cell culture is maintained at greater than about 0° C. In some embodiments, said cell culture is maintained at about 2° C. to about 8° C.

### Cell Passaging

Provided herein, in one aspect, is a method for preparing stem cells for infusion, the method comprising:
a. providing a cryopreserved population of cells comprising said stem cells;
b. warming said stem cells to a first temperature and holding said stem cells at said first temperature for a first period of time; and
c. changing said first temperature to a second temperature and maintaining said stem cells at said second temperature for a period of time.

In some embodiments, for a method provided herein, prior to (a), said stem cells were passaged, for example, during the culturing methods described herein. In some embodiments, for a method provided herein, prior to (a), said stem cells were passaged at least one time, 2 times, 3 times, 4 times, 5 times, 6 times, 7 times, 8 times, 9 times, or 10 times. In some embodiments, for a method provided herein, prior to (a), said stem cells were passaged at least one time. In some embodiments, for a method provided herein, prior to (a), said stem cells were passaged at least 2 times. In some embodiments, for a method provided herein, prior to (a), said stem cells were passaged at least 4 times. In some embodiments, for a method provided herein, prior to (a), said stem cells were passaged one time, 2 times, 3 times, 4 times, 5 times, 6 times, 7 times, 8 times, 9 times, or 10 times. In some embodiments, for a method provided herein, prior to (a), said stem cells were passaged more than 10 times.

Provided herein, in another aspect, is a method for preparing a sample of stem cells for direct infusion, the method comprising:
a. providing a cell culture comprising said sample of stem cells wherein said cell culture was cryopreserved and passaged;
b. thawing said cell culture; and
c. maintaining said cell culture at less than 40° C.

In some embodiments, said cell culture was passaged. In some embodiments, said cell culture was passaged at least one time, 2 times, 3 times, 4 times, 5 times, 6 times, 7 times, 8 times, 9 times, or 10 times. In some embodiments, said cell culture was passaged at least one time. In some embodiments, said cell culture was passaged at least 2 times. In some embodiments, said cell culture was passaged at least 4 times. In some embodiments, said cell culture was passaged one time, 2 times, 3 times, 4 times, 5 times, 6 times, 7 times, 8 times, 9 times, or 10 times.

### Cell Culturing and Packaging

In some embodiments, for a method provided herein, the method further comprises, prior to (c), culturing said stem cells at about 10,000 to about 50,000, about 10,000 to about 40,000, about 10,000 to about 30,000, about 10,000 to about 20,000, about 20,000 to about 50,000, about 20,000 to about 40,000, about 20,000 to about 30,000, about 30,000 to about 50,000, about 30,000 to about 40,000, or about 40,000 to about 50,000 cells/cm² at said first temperature. In some embodiments, for a method provided herein, the method further comprises, prior to (c), culturing said stem cells at about 10,000 cells/cm² to about 50,000 cells/cm² at said first temperature. In some embodiments, for a method provided herein, the method further comprises, prior to (c), culturing said stem cells at about 10,000, about 20,000, about 30,000, about 40,000, or about 50,000 cells/cm² at said first temperature. In some embodiments, for a method provided herein, the method further comprises, prior to (c), culturing said stem cells at about 32,000 cells/cm² at said first temperature.

In some embodiments, for a method provided herein, the method further comprises, prior to (c), packaging said stem cells in a volume comprising about 10×10² to about 10×10¹⁰, about 10×10² to about 10×10⁹, about 10×10² to about 10×10⁸, about 10×10² to about 10×10⁷, about 10×10² to about 10×10⁶, about 10×10² to about 10×10⁵, about 10×10² to about 10×10⁴, about 10×10² to about 10×10³, about 10×10³ to about 10×10¹⁰, about 10×10³ to about 10×10⁹, about 10×10³ to about 10×10⁸, about 10×10³ to about 10×10⁷, about 10×10³ to about 10×10⁶, about 10×10³ to about 10×10⁵, about 10×10³ to about 10×10⁴, about 10×10⁴ to about 10×10¹⁰, about 10×10⁴ to about 10×10⁹, about 10×10⁴ to about 10×10⁸, about 10×10⁴ to about 10×10⁷, about 10×10⁴ to about 10×10⁶, about 10×10⁴ to about 10×10⁵, about 10×10⁵ to about 10×10¹⁰, about 10×10⁵ to about 10×10⁹, about 10×10⁵ to about 10×10⁸, about 10×10⁵ to about 10×10⁷, about 10×10⁵ to about 10×10⁶, about 10×10⁶ to about 10×10¹⁰, about 10×10⁶ to about 10×10⁹, about 10×10⁶ to about 10×10⁸, about 10×10⁶ to about 10×10⁷, about 10×10⁷ to about 10×10¹⁰, about 10×10⁷ to about 10×10⁹, about 10×10⁷ to about 10×10⁸, about 10×10⁸ to about 10×10¹⁰, about 10×10⁸ to about 10×10⁹, or about 10×10⁹ to about 10×10¹⁰ live cells/mL and maintaining said stem cells at said second temperature. In some embodiments, for a method provided herein, the method further comprises, prior to (c), packaging said stem cells in a volume comprising about 10×10² live cells/mL to about 10×10¹⁰ live cells/mL and maintaining said stem cells at said second temperature. In some embodiments, for a method provided herein, the method further comprises, prior to (c), packaging said stem cells in a volume comprising about 10×10⁶ live cells/mL and maintaining said stem cells at said second temperature.

In some embodiments, for a method provided herein, the method further comprises, prior to (c), packaging said stem cells in a volume comprising less than 10 mL, 9 mL, 8 mL, 7 mL, or 6 mL. In some embodiments, for a method provided herein, the method further comprises, prior to (c), packaging said stem cells in a volume comprising 10 mL, 9 mL, 8 mL, 7 mL, 6 mL, 5 mL, 4 mL, 3 mL, 2 mL, or 1 mL. In some embodiments, for a method provided herein, the method further comprises, prior to (c), packaging said stem cells in a volume comprising 5 mL.

### Cell Viability

In some embodiments, for a method provided herein, said sample of stem cells comprises at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% viable cells after (c). In some embodiments, for a method provided herein, said sample of stem cells comprises about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% viable cells after (c). In some embodiments, for a method provided herein, said sample of stem cells comprises at least about 70% viable cells after (c). In some embodiments, for a method provided herein, said sample of stem cells comprises about 70% viable cells after (c).

Provided herein, in another aspect, is a method for preparing a sample of stem cells for direct infusion, the method comprising:
a. providing a cell culture comprising said sample of stem cells wherein said cell culture was cryopreserved and passaged;
b. thawing said cell culture; and
c. maintaining said cell culture at less than room temperature;
   wherein said sample of stem cells comprises at least about 70% viable cells.

In some embodiments, for a method provided herein, said sample of stem cells comprises at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% viable cells after (c). In some embodiments, for a method provided herein, said sample of stem cells comprises about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% viable cells after (c). In some embodiments, for a method provided herein, said sample of stem cells comprises at least about 70% viable cells after (c). In some embodiments, for a method provided herein, said sample of stem cells comprises about 70% viable cells after (c).

### Cryopreservation and Rinse Media

In some embodiments, for a method provided herein, said cell culture was cryopreserved in a cryopreservation media, wherein said cryopreservation media comprises an electrolyte formulation, human serum albumin (HSA), dimethyl sulfoxide (DMSO), or any combination thereof.

In some embodiments, said cryopreservation media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% HSA. In some embodiments, said cryopreservation media comprises about 1% to about 5% HSA. In some embodiments, said cryopreservation media comprises about 2.5% HSA.

In some embodiments, said cryopreservation media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% DMSO. In some embodiments, said cryopreservation media comprises about 1% to about 10% DMSO. In some embodiments, said cryopreservation media comprises about 5% DMSO.

In some embodiments, said electrolyte formulation is Plasmalyte A.

In some embodiments, for a method provided herein, the method further comprises, prior to (c), resuspending said cell culture in a rinse media, wherein said rinse media comprises an electrolyte formulation, human serum albumin (HSA), or both. In some embodiments, for a method provided herein, the method further comprises, prior to (c), resuspending said cell culture in a rinse media, wherein said rinse media comprises an electrolyte formulation, human serum albumin (HSA), or both.

In some embodiments, the rinse media is fresh.

In some embodiments, said rinse media comprises about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% HSA. In some embodiments, said rinse media comprises about 1% to about 5% HSA. In some embodiments, said rinse media comprises about 2.5% HSA.

In some embodiments, said electrolyte formulation is Plasmalyte A.

### Antigens of Hypothermically Stored Mesenchymal Stem Cells

Provided herein, in another aspect, is a method for preparing a sample of stem cells for direct infusion, the method comprising:
a. providing a cell culture comprising said sample of stem cells wherein said cell culture was cryopreserved and passaged;
b. thawing said cell culture; and
c. maintaining said cell culture at less than room temperature;
   wherein said sample of stem cells comprises less than about 1% to about 10% CD45+ cells.

In some embodiments, said sample of stem cells comprises less than about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% CD45+ cells. In some embodiments, said sample of stem cells comprises less than about 1% to about 10% CD45+ cells. In some embodiments, said sample of stem cells comprises less than about 5% CD45+ cells.

In some embodiments, said sample of stem cells comprises less than about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 10%, about 2% to about 9%, about 2% to about 8%, about 2% to about 7%, about 2% to about 6%, about 2% to about 5%, about 2% to about 4%, about 2% to about 3%, about 3% to about 10%, about 3% to about 9%, about 3% to about 8%, about 3% to about 7%, about 3% to about 6%, about 3% to about 5%, about 3% to about 4%, about 4% to about 10%, about 4% to about 9%, about 4% to about 8%, about 4% to about 7%, about 4% to about 6%, about 4% to about 5%, about 5% to about 10%, about 5% to about 9%, about 5% to about 8%, about 5% to about 7%, about 5% to about 6%, about 6% to about 10%, about 6% to about 9%, about 6% to about 8%, about 6% to about 7%, about 7% to about 10%, about 7% to about 9%, about 7% to about 8%, about 8% to about 10%, about 8% to about 9%, or about 9% to about 10% CD34+ cells. In some embodiments, said sample of stem cells comprises less than about 1% to about 10% CD34+ cells. In some embodiments, said sample of stem cells comprises less than about 5% CD34+ cells.

Provided herein, in another aspect, is a method for preparing a sample of stem cells for direct infusion, the method comprising:

a. providing a cell culture comprising said sample of stem cells wherein said cell culture was cryopreserved and passaged;
b. thawing said cell culture; and
c. maintaining said cell culture at less than room temperature;
wherein said sample of stem cells comprises at least about 90% to about 99% CD90+ cells.

In some embodiments, said sample of stem cells comprises at least about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 99%, about 91% to about 98%, about 91% to about 97%, about 91% to about 96%, about 91% to about 95%, about 91% to about 94%, about 91% to about 93%, about 91% to about 92%, about 92% to about 99%, about 92% to about 98%, about 92% to about 97%, about 92% to about 96%, about 92% to about 95%, about 92% to about 94%, about 92% to about 93%, about 93% to about 99%, about 93% to about 98%, about 93% to about 97%, about 93% to about 96%, about 93% to about 95%, about 93% to about 94%, about 94% to about 99%, about 94% to about 98%, about 94% to about 97%, about 94% to about 96%, about 94% to about 95%, about 95% to about 99%, about 95% to about 98%, about 95% to about 97%, about 95% to about 96%, about 96% to about 99%, about 96% to about 98%, about 96% to about 97%, about 97% to about 99%, about 97% to about 98%, or about 98% to about 99% CD90+ cells. In some embodiments, said sample of stem cells comprises at least about 90% to about 99% CD90+ cells. In some embodiments, said sample of stem cells comprises at least about 95% CD90+ cells.

Provided herein, in another aspect, is a method for preparing a sample of stem cells for direct infusion, the method comprising:

a. providing a cell culture comprising said sample of stem cells wherein said cell culture was cryopreserved and passaged;
b. thawing said cell culture; and
c. maintaining said cell culture at less than room temperature;
wherein said sample of stem cells comprises at least about 90% to about 99% CD105+ cells.

In some embodiments, said sample of stem cells comprises at least about 90% to about 99%, about 90% to about 98%, about 90% to about 97%, about 90% to about 96%, about 90% to about 95%, about 90% to about 94%, about 90% to about 93%, about 90% to about 92%, about 90% to about 91%, about 91% to about 99%, about 91% to about 98%, about 91% to about 97%, about 91% to about 96%, about 91% to about 95%, about 91% to about 94%, about 91% to about 93%, about 91% to about 92%, about 92% to about 99%, about 92% to about 98%, about 92% to about 97%, about 92% to about 96%, about 92% to about 95%, about 92% to about 94%, about 92% to about 93%, about 93% to about 99%, about 93% to about 98%, about 93% to about 97%, about 93% to about 96%, about 93% to about 95%, about 93% to about 94%, about 94% to about 99%, about 94% to about 98%, about 94% to about 97%, about 94% to about 96%, about 94% to about 95%, about 95% to about 99%, about 95% to about 98%, about 95% to about 97%, about 95% to about 96%, about 96% to about 99%, about 96% to about 98%, about 96% to about 97%, about 97% to about 99%, about 97% to about 98%, or about 98% to about 99% CD105+ cells. In some embodiments, said sample of stem cells comprises at least about 90% to about 99% CD105+ cells. In some embodiments, said sample of stem cells comprises at least about 95% CD105+ cells.

### Fibroblast Colony-Forming Units (CFU-Fs) of Hypothermically Stored Mesenchymal Stem Cells

Provided herein, in another aspect, is a method for preparing a sample of stem cells for direct infusion, the method comprising:
a. providing a cell culture comprising said sample of stem cells wherein said cell culture was cryopreserved and passaged;
b. thawing said cell culture; and
c. maintaining said cell culture at less than room temperature;
   wherein said sample of stem cells comprises at least about 10,000 to about 300,000 CFU-F/1 million viable cells.

In some embodiments, said sample of stem cells comprises at least about 10,000 to about 300,000, about 10,000 to about 250,000, about 10,000 to about 200,000, about 10,000 to about 150,000, about 10,000 to about 100,000, about 10,000 to about 50,000, about 10,000 to about 40,000, about 10,000 to about 30,000, about 10,000 to about 20,000, about 20,000 to about 300,000, about 20,000 to about 250,000, about 20,000 to about 200,000, about 20,000 to about 150,000, about 20,000 to about 100,000, about 20,000 to about 50,000, about 20,000 to about 40,000, about 20,000 to about 30,000, about 30,000 to about 300,000, about 30,000 to about 250,000, about 30,000 to about 200,000, about 30,000 to about 150,000, about 30,000 to about 100,000, about 30,000 to about 50,000, about 30,000 to about 40,000, about 40,000 to about 300,000, about 40,000 to about 250,000, about 40,000 to about 200,000, about 40,000 to about 150,000, about 40,000 to about 100,000, about 40,000 to about 50,000, about 50,000 to about 300,000, about 50,000 to about 250,000, about 50,000 to about 200,000, about 50,000 to about 150,000, about 50,000 to about 100,000, about 100,000 to about 300,000, about 100,000 to about 250,000, about 100,000 to about 200,000, about 100,000 to about 150,000, about 150,000 to about 300,000, about 150,000 to about 250,000, about 150,000 to about 200,000, about 200,000 to about 300,000, about 200,000 to about 250,000, or about 250,000 to about 300,000 CFU-F/1 million viable cells. In some embodiments, said sample of stem cells comprises at least about 10,000 to about 300,000 CFU-F/1 million viable cells. In some embodiments, said sample of stem cells comprises at least about 10,000 CFU-F/1 million viable cells.

### Types of Stem Cells

In some embodiments, for a method provided herein, said sample of stem cells comprise vertebral bone adherent mesenchymal stem cells (vBA-MSCs), vertebral body bone marrow mesenchymal stem cells (vBM-MSCs), or both. In some embodiments, for a method provided herein, said sample of stem cells comprise vertebral bone adherent mesenchymal stem cells (vBA-MSCs). In some embodiments, for a method provided herein, said sample of stem cells comprise vertebral body bone marrow mesenchymal stem cells (vBM-MSCs). In some embodiments, for a method provided herein, said sample of stem cells comprise both vertebral bone adherent mesenchymal stem cells (vBA-MSCs) and vertebral body bone marrow mesenchymal stem cells (vBM-MSCs).

Provided herein, in another aspect, is a method for preparing a sample of vertebral bone adherent mesenchymal stem cells (vBA-MSCs) for direct infusion, the method comprising:
a. providing a cell culture comprising said sample of stem cells wherein said cell culture was cryopreserved and passaged;
b. thawing said cell culture; and
c. maintaining said cell culture at less than room temperature.

In some embodiments, for a method provided herein, said stem cells are derived from a cadaver.

### Definitions

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

As used herein, the term “passaging” refers to removing the media from a cell culture and transferring the cells into fresh growth medium. Passaging allows for the further propagation of the cell line.

### EXAMPLES

### Example 1: Mesenchymal Stem Cell (MSC) Recovery Following Cryopreservation

Passage 2 (P2) mesenchymal stem cells (MSCs) from 3 donors were cryopreserved in 5% Freeze Media (Plasmalyte A+2.5% Human Serum Albumin+5% DMSO) in 2 mL cryovials as per standard protocol. Vials were removed from liquid nitrogen vapor storage and rapidly thawed in a 37° C. water bath. Cells were diluted with 1 volume of Rinse Media (Plasmalyte A+2.5% Human Serum Albumin), centrifuged, resuspended, and plated in T25 flasks at 32,000 cells/cm² or 800,000 cells/T25 (which is the equivalent density of a 100M cell end of production (EOP) in a 5-chamber CellSTACK). Cells were lifted from the first set of flasks at 24 hours. Cells were lifted from the second set of flasks at 48 hours. A cell count was taken after lifting, and cells were plated for fibroblast colony-forming units (CFU-F). CFU-F was stained after 10 days with crystal violet and colonies containing 50 cells or more were enumerated.

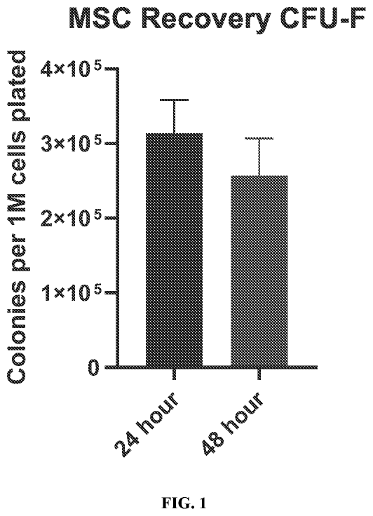

The 24 hour data is listed in Table 2 while the 48 hour data is listed in Table 3. Data includes donors, Live Cell/mL count, Total Cell/mL count, % Recovered, and CFU-F results. FIG. 1 shows the CFU-F data in graph form.

TABLE 2
24 Hour MSC Culture Recovery

| Donor | Live Cells | Total Cells | % Recovered | P3 CFU-F/1M cells |
|---|---|---|---|---|
| 1 | 1.45 × 10⁶ | 1.65 × 10⁶ | 181% | 2.70 × 10⁵ |
| 2 | 7.81 × 10⁵ | 9.42 × 10⁵ | 98% | 3.10 × 10⁵ |
| 3 | 9.44 × 10⁵ | 1.09 × 10⁶ | 118% | 3.60 × 10⁵ |

TABLE 3
48 Hour MSC Culture Recovery

| Donor | Live Cells | Total Cells | % Recovered | P3 CFU-F/1M cells |
|---|---|---|---|---|
| 1 | 2.29 × 10⁶ | 2.58 × 10⁶ | 286% | 2.50 × 10⁵ |
| 2 | 1.84 × 10⁶ | 1.97 × 10⁶ | 230% | 2.10 × 10⁵ |
| 3 | 1.94 × 10⁶ | 2.24 × 10⁶ | 242% | 3.10 × 10⁵ |

These data indicate that logarithmic expansion post-thaw begins between 24 and 48 hours.

The cells are then packaged at 5 mL volumes of 10×10⁶ viable P4 vBM-MSC/mL in Plasma-Lyte A+2.5% HSA (Rinse Media) in CellSeal® 5 mL cryovials (Sexton Biotechnologies, Indianapolis, Ind.). The vials are placed on wet ice, resulting in hypothermic conditions (e.g. about 2° C. to 8° C. Once packaged with the wet ice, the cells are shipped to the recipient. Shipping can take any time between 30 minutes to 2 days or more.

### Example 2: Recovery Following Cryopreservation of Vertebral Bone Adherent Mesenchymal Stem Cells (vBA-MSCs) Versus Vertebral Body Bone Marrow Mesenchymal Stem Cells (vBM-MSCs)

Vertebral bone adherent mesenchymal stem cells (vBA-MSCs) and vertebral body bone marrow mesenchymal stem cells (vBM-MSCs) are cryopreserved in 5% Freeze Media (Plasmalyte A+2.5% Human Serum Albumin+5% DMSO) in 2 mL cryovials as per standard protocol. Vials are removed from liquid nitrogen vapor storage and rapidly thawed in a 37° C. water bath. Cells are diluted with 1 volume of Rinse Media (Plasmalyte A+2.5% Human Serum Albumin), centrifuged, resuspended, and plated in T25 flasks at 32,000 cells/cm² or 800,000 cells/T25 (which is the equivalent density of a 100M cell end of production (EOP) in a 5-chamber CellSTACK). Cells are lifted from the first set of flasks at 24 hours. Cells are lifted from the second set of flasks at 48 hours. A cell count is taken after lifting, and cells are plated for fibroblast colony-forming units (CFU-F). CFU-F are stained after 10 days with crystal violet and colonies containing 50 cells or more are enumerated.

It is anticipated that vertebral bone adherent mesenchymal stem cells (vBA-MSCs) result in a higher number of fibroblast colony-forming units (CFU-F) than vertebral body bone marrow mesenchymal stem cells (vBM-MSCs).

### Example 3: Protocol for Hypothermic Storage and Administration of Vertebral Body Mesenchymal Stem Cells

Cryo-recovered passage 4 (P4) Vertebral Body Bone Marrow Mesenchymal Stem Cells (vBM-MSCs) are produced from human cadaveric organ and tissue donors and packaged at 5 mL volumes of 10×10⁶ viable P4 vBM-MSC/mL in Plasma-Lyte A+2.5% HSA (Rinse Media) in CellSeal® 5 mL cryovials (Sexton Biotechnologies, Indianapolis, Ind.). This product and packaging configuration is designed for direct injection with no further manipulation required at the clinical site.

After receipt at the clinical site, cryo-recovered P4 vBM-MSC vials at stored at hypothermic temperatures (2 to 8° C.) in the Cryoport C3 shipper until ready for patient administration. The cooler lid and phase change panel from the Cryoport C3 shipper are removed, and the CellSeal® vials are removed from the Cryoport C3 shipper. The vials are inspected for damage and labels are ensured to be legible and still adhered.

When the patient is ready for injection, one recovered P4 vBM-MSC vial is removed from hypothermic storage. The vial is gently inverted a few times to mix well and placed in a biosafety cabinet upright with the tubing and port at the top in a processing rack. The foil is removed from the bottom of the retrieval port, and the retrieval port septum is thoroughly swabbed with a sterile alcohol pad and allowed to air dry up to one minute before accessing. A 5 mL syringe is preloaded with 2 mL of air. The syringe is attached to the Vial Adapter via the luer lock connection. The preloaded air is slowly expelled to ensure the leur-activated valve is open.

The CellSeal® vial septum is punctured using a steady force ensuring the spike enters the septum perpendicular. The CellSeal® vent tubing is cut open with clean scissors, using caution to ensure the fill tubing is not also cut. The Vial Adapter body is gripped and the syringe plunger is gradually withdrawn until the sample is retrieved. The Vial Adapter body is gripped and the syringe is disconnected. The CellSeal® vial and Vial Adapter are disposed of. An injection needle is attached to the syringe, and the sample is administered as per clinical protocol. A second vial is administered utilizing the same steps as the first vial. Two vials are equivalent to one dose.

### Example 4: Recovery, Storage, and Shipping of Vertebral Body Mesenchymal Stem Cells

Vertebral bone adherent mesenchymal stem cells (vBA-MSCs) and vertebral body bone marrow mesenchymal stem cells (vBM-MSCs) are recovered from one or more cadavers as described herein and in PCT/US2021/055066 filed on Oct. 14, 2021, which hereby incorporated by reference in its entirety.

Upon recovery, the MSC populations are cryopreserved as described herein and in PCT/US2021/055066. Whilst in cryopreservation, the MSCs are stored in one cell bank storage center of a plurality of cell bank storage centers located at distinct geographical locations.

Upon a need for a MSC infusion/administration to a patient in need thereof, an attending physician and/or the physician's employer contacts the networks of cell bank storage centers to identify a recommended MSC populations based on specific qualities of the patent in need thereof, e.g. human leukocyte antigen (HLA) profile.

Upon identification of a recommend MSC population, the sample comprising the recommended MSC population is held at a first temperature greater than 0° C. to thaw. Once thawed, the recommended MSC population is resuspended in the Alpha-MEM media described in Table 1. The recommended MSC population is then plated in a cell storage medium, in the Alpha-MEM medium, at about room temperature at about 32,000 cells/cm² of cell storage medium substrate. The recommended MSC population is cultured for 24-48 hours under conditions as to remain less than about 70-90% confluency of the cell storage medium substrate and allowed to undergo between zero and no more than 3 population doublings (e.g. Example 2 described herein).

Finally, the recommended MSC population is packaged and shipped, e.g. according to Example 3 described herein, i.e. the recommended MSC population is maintained at a second temperature that is hypothermic. The recommended MSC population is then administered to the patient in need thereof as described in Example 3.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for preparing stem cells for infusion, the method comprising:
a. providing a cryopreserved population of cells comprising said stem cells;
b. warming said stem cells to a first temperature greater than 0° C. and holding said stem cells at said first temperature for a first period of time; and
c. changing said first temperature to a second temperature less than 40° C. and maintaining said stem cells at said second temperature for a period of time;
wherein said stem cells comprise at least about 10,000 to about 300,000 CFU-F/1 million viable cells.

2. The method of claim 1, wherein said second temperature is hypothermic.

3. The method of claim 1, wherein said first period of time or said time period is less than one week.

4. The method of claim 3, wherein said first period of time or said time period is less than 5 days.

5. The method of claim 4, wherein said first period of time or said time period is less than 1 day.

6. The method of claim 5, wherein said first period of time or said time period is less than 12 hours.

7. The method of claim 1, wherein, prior to (a), said stem cells were passaged at least one time.

8. The method of claim 7, wherein, prior to (a), said stem cells were passaged at least 2 times.

9. The method of claim 8, wherein, prior to (a), said stem cells were passaged at least 4 times.

10. The method of claim 1, further comprising, prior to (c), culturing said stem cells at about 10,000 cells/cm² to about 50,000 cells/cm² at said first temperature.

11. The method of claim 10, further comprising, prior to (c), culturing said stem cells at about 32,000 cells/cm² at said first temperature.

12. The method of claim 1, further comprising packaging said stem cells in a volume comprising about 10×10² live cells/mL to about 10×10¹⁰ live cells/mL.

13. The method of claim 12, further comprising packaging said stem cells in a volume comprising about 10×10⁶ live cells/mL.

14. The method of claim 1, further comprising packaging said stem cells in a volume comprising less than 10 mL, 9 mL, 8 mL, 7 mL, or 6 mL.

15. The method of claim 14, further comprising packaging said stem cells in a volume comprising 5 mL.

16. The method of claim 1, wherein said first time period is a time period wherein said stem cells do not double.

17. The method of claim 1, wherein said first time period is a time period wherein said stem cells undergo one or more population doublings.

18. The method of claim 1, wherein said stem cells are maintained to less than 80% confluency relative to a cell culture storage medium during said first time period or said second time period.

19. The method of claim 1, wherein said stem cells comprise vertebral bone adherent mesenchymal stem cells (vBA-MSCs), vertebral body bone marrow mesenchymal stem cells (vBM-MSCs), or both.

20. The method of claim 1, wherein said stem cells are derived from a cadaver.

21. The method of claim 1, wherein said stem cells comprise at least about 90,000 CFU-F/1 million viable cells.

22. The method of claim 1, wherein said stem cells comprise at least about 70% viable cells.
